(19) United States
(12) Patent Application Publication
(10) Pub. No.: US 2024/0393337 A1
(43) Pub. Date: Nov. 28, 2024

(54) Lung Cancer Prediction and Uses Thereof

(71) Applicant: SomaLogic Operating Co., Inc., Boulder, CO (US)

(72) Inventors: Leigh Alexander, Boulder, CO (US); Clare Paterson, Boulder, CO (US); Rachel M. Ostroff, Boulder, CO (US); Yolanda Hagar, Boulder, CO (US); Natasha Kureshi, Boulder, CO (US)

(73) Assignee: SomaLogic Operating Co., Inc., Boulder, CO (US)

(21) Appl. No.: 18/693,210

(22) PCT Filed: Oct. 7, 2022
(86) PCT No.: PCT/US2022/045989

§ 371 Date: Mar. 19, 2024

Related U.S. Application Data
(60) Provisional application No. 63/253,509, filed on Oct. 7, 2021.

(51) Int. Cl. G01N 33/574; G01N 33/68

(52) U.S. Cl. CPC G01N 33/57423; G01N 33/6893; G01N 2333/715; G01N 2333/785; G01N 2333/91102; G01N 2333/96419

(57) ABSTRACT

The present disclosure includes biomarkers, methods, devices, reagents, systems, and kits for evaluating and predicting lung cancer risk. In one aspect, the disclosure provides biomarkers that can be used alone or in various combinations to estimate or determine lung cancer risk. In another aspect, methods are provided for evaluating and predicting lung cancer risk in an individual, where the methods include detecting, in a biological sample from an individual, at least one biomarker value corresponding to at least one biomarker selected from the group of biomarkers provided in Table 6.

### CROSS-REFERENCE TO RELATED APPLICATIONS

This application claims the benefit of priority of U.S. Provisional Application No. 63/253,509, filed Oct. 7, 2021, which is incorporated by reference herein in its entirety for any purpose.

### FIELD OF THE INVENTION

The present application relates generally to the detection of biomarkers and methods of evaluating the risk of lung cancer in an individual and, more specifically, to one or more biomarkers, methods, devices, reagents, systems, and kits used to assess an individual for the prediction of risk of developing lung cancer within a specified time frame.

### BACKGROUND

The following description provides a summary of information relevant to the present application and is not an admission that any of the information provided or publications referenced herein is prior art to the present application.

Lung cancer is the second most common cancer type and is the leading cause of cancer death in both men and women in the U.S. (Siegel et al. “Cancer Statistics, 2021.” CA Cancer J Clin 2021; 71:7-33).

There are two main categories of lung cancer which are classified according to cell type, immunohistochemical, and molecular characteristics 1) non-small cell lung cancer, which includes squamous cell carcinoma, large cell carcinoma and adenocarcinoma and accounts for around 85% of lung cancers, and 2) small cell lung cancer (which includes small cell carcinoma and combined small cell carcinoma). Small cell lung cancer is faster growing, and around 70% of individuals with this cancer type will have cancer that has already spread by the time of diagnosis. (“About Lung Cancer”, American Cancer Society. Available online at https://www.cancer.org/cancer/lung-cancer/about/and “Cancer Stat Facts: Lung and Bronchus Cancer” National Cancer Institute. Surveillance, Epidemiology, and End Results Program. Available online at https://seer.cancer.gov/statfacts/html/lungb.html).

Lung cancer patients can present at varying stages of illness, and initial symptoms are generally observed as a persistent cough, shortness of breath, and blood present in the sputum. The diagnosis of lung cancer is based upon the initial presence of lung nodules found via chest imaging (low dose computed tomography is the gold standard but in some clinical settings may also include chest radiography or MRI as alternatives), followed by biopsy.

The size, invasiveness, and spread of the tumor to lymph nodes determines the stage of lung cancer. Lung cancer has a poor prognosis that worsens with increasing stage progression. Patients with localized lung cancer stages have a 59% 5-year survival rate, which decreases to a 32% 5-year survival rate for those with lung cancer stages associated with regional spread, and 6% 5-year survival rate for those with distant metastases. (https://seer.cancer.gov/statfacts/html/lungb.html).

There are many different types of treatments for patients with lung cancer dependent upon cancer stage and overall health, including surgery, radiation therapy, chemotherapy, targeted therapies, and immunotherapy. (“Non-Small Cell Lung Cancer Treatment (PDQ®)-Patient Version” (August 2021) National Cancer Institute. Available online at https://www.cancer.gov/types/lung/patient/non-small-cell-lung-treatment-pdq#_118).

Around 1 in 15 men and 1 in 17 women will develop lung cancer in their lifetime. (https://seer.cancer.gov/statfacts/html/lungb.html). However, that risk dramatically increases in individuals who smoke, with 80% or more lung cancer cases occurring in the United States in people who are smokers. (“Lung Cancer Among People Who Never Smoked” (November 2020) Center for Disease Control and Prevention. Available online at https://www.cdc.gov/cancer/lung/nonsmokers/index.htm). Lung cancer risk in both male and female smokers increases with the cumulative quantity and duration of smoking (defined in “pack years”) and decreases with increased time since quitting in former smokers (Bruder et al. “Estimating lifetime and 10-year risk of lung cancer.” Prev Med Rep 2018; 11:125-30 and Samet J. M. “Health benefits of smoking cessation.” Clin Chest Med 1991; 12:669-79 and (FIG. 1).

While smoking is well demonstrated as the most penetrant risk factor for lung cancer, increasing age is also a significant lung cancer risk with the median age of lung cancer diagnosis being 71, and being most frequently diagnosed amongst people aged 65-74. https://seer.cancer.gov/statfacts/html/lungb.html.

Additionally, other risk factors including exposure to smoke, workplace chemicals (e.g. asbestos), and radiation, as well as clinical factors such as having a family history of lung cancer have been linked to elevating an individual's risk for lung cancer or lung disease. https://www.cancer.gov/types/lung/patient/non-small-cell-lung-treatment-pdq#_118.

The United States Preventative services Task Force (USPSTF) recommends with moderate certainty (Grade B rating) that annual screening with low dose computed tomography (LDCT) has a moderate net benefit in individuals considered at high risk of lung cancer. High risk individuals are defined as those aged 50-80 years old, who have at least a 20 pack-year smoking history and currently smoke or have quit within the past 15 years. (Force USPST, et al. “Screening for Lung Cancer: US Preventive Services Task Force Recommendation Statement.” JAMA 2021;325:962-70).

These screening guidelines have been restricted to high risk individuals (based on age and cigarette smoking status) due to sufficient evidence accumulating that annual LDCT screening reduces lung cancer mortality in high risk individuals. For example, The National Lung Cancer Screening Trial (NLST) is a randomized controlled trial which recruited individuals at high-risk for lung cancer under previous USPSTF guidelines (individuals aged 55-80, who have at least a 30 pack-year smoking history and currently smoke or have quit within the past 15 years) and compared the effectiveness of LDCT scans compared to chest radiography. The study reported a 20% reduction in lung cancer mortality with LDCT screening which was significantly superior to chest radiography. (National Lung Screening Trial Research Team. “Reduced lung-cancer mortality with low-dose computed tomographic screening.” N Engl J Med 2011;365:395-409). In the event of abnormal findings with LDCT, subsequent screening for lung cancer is often modified to more frequent LDCT according to Lung-RADS assessment categories. (“Lung CT Screening Reporting & Data System (Lung-RADS)” American College of Radiology, Available online at https://www.acr.org/Clinical-Resources/Reporting-and-Data-Systems/Lung-Rads.)

The USPSTF does not recommend lung cancer screening to lower risk individuals (i.e., non-smokers) because there is not sufficient evidence for net benefit in this population, and the risk of harms of screening (including false-positive results leading to unnecessary tests and invasive procedures, overdiagnosis, radiation-induced cancer, incidental findings, and increases in distress or anxiety) outweigh the benefits in lower risk populations. However, some healthcare systems may, under physician guidance, recommend routine lung cancer screening to non-eligible individuals who are lung cancer survivors, have a strong family history of lung cancer, or those who have been exposed to occupational asbestos. (“Lung Cancer Screening” (March 2021) Mayo Clinic. Available online at https://www.mayoclinic.org/tests-procedures/lung-cancer-screening/about/pac-20385024). Reimbursement for screening in these individuals is not assured.

In addition to LDCT, screening for lung cancer can also be performed via chest radiography, sputum cytology and biomarker measurements, however; the evidence for these screening modalities to bestow mortality benefits is insufficient and these technologies come with a lower sensitivity than LDCT. (Force USPST, et al. “Screening for Lung Cancer: US Preventive Services Task Force Recommendation Statement.” JAMA 2021;325:962-70). Moreover, following an abnormal finding in one of these alternative modalities of lung cancer screening would lead to the recommendation of a follow up LDCT screening in order to achieve mortality benefits.

Lung cancer screening is a joint decision-making process between the patient and provider that should take place in addition to smoking cessation counseling (in current smokers). The risks, benefits, and evidence level of each screening modality should be discussed, as well as advice on where screening should be conducted (High-quality lung cancer and treatment center that employs Lung-RADS standardized categorization). In cases where the risk of lung cancer is particularly high (e.g., screening eligible current heavy smoker, with history of COPD and a strong family history of lung cancer), the physician may modify guidance to strongly suggest LDCT as a screening modality (as it is the gold standard), and screening to be performed at a Screening Center of Excellence (to ensure the highest level of sensitivity and specificity is achieved).

While it is well established that lung cancer screening with LDCT benefits reduce lung cancer mortality rates, available data indicate that uptake of lung cancer screening is low with studies showing that as few as 14% of individuals eligible for lung cancer screening had been screened in the previous years. (Zahnd et al. “Lung Cancer Screening Utilization: A Behavioral Risk Factor Surveillance System Analysis.” Am J Prev Med 2019;57:250-5). With an estimated over 130 thousand people in the US projected to die of lung cancer in 2021, (https://seer.cancer.gov/statfacts/html/lungb.html) additional clinical care paths to advise patients' risk for lung cancer are needed. There is no current clinically accepted standard of care for assessing a patient's risk for future lung cancer diagnosis.

As described above, physicians may recommend differing lung cancer screening modalities or referral to a Screening Center of Excellence for lung cancer based on individual patients' risk level; however, screening tools are limited to detecting current lung cancer, not future risk. A variety of clinical risk calculators for future lung risk have been developed that can predict an individual's lung cancer risk from a combination of demographic, personal and family health history, lifestyle, and carcinogen exposure level; however, these calculators are routinely not validated/replicated in independent cohorts, involve patient self-report information, and many require non-standard clinical outputs. No clinical risk calculators are currently widely used as standard of care in clinical practice. Accordingly, a need exists for biomarkers, methods, devices, reagents, systems, and kits to evaluate an individual's lung cancer risk.

### SUMMARY OF THE INVENTION

The present application discloses biomarkers, methods, devices, reagents, systems, and kits to evaluate an individual's risk for lung cancer diagnosis within a specified time frame. In one aspect, the objective of the presently disclosed lung cancer risk test is to create a model that predicts a current or former smoker's risk for a lung cancer diagnosis within 5 years of blood draw.

Benefits of the presently disclosed lung cancer risk test include: a convenient way to gain personalized knowledge of the degree of risk for a future lung cancer diagnosis without reliance on self-reported demographics or genetic background information; the test result may influence compliance with lung cancer screening guidelines allowing for the potential for earlier identification of lung cancer, and thus improved chance of lung cancer survival; the test may influence positive behavior changes in modifiable risk-related behaviors (e.g., smoking cessation, dietary change, weight loss); and the test may aid healthcare provider lung cancer screening decisions/recommendations or patient's lung screening preferences based on the test result (e.g., for patients in high-risk category to initially undertake LDCT screening methodology that is considered the gold standard versus another lesser sensitive methodology such as chest radiography). The lung cancer risk test may include identifying subjects that have lung cancer at the time of sampling.

The following numbered paragraphs-contain statements of broad combinations of the inventive technical features herein disclosed:

1. A method comprising:
a) measuring the level of PSP-94 protein and the level of at least one, two, three, four, five, or six proteins selected from the group consisting of MMP-12, SP-D, HE4, PH, FUT5 and CRLF1 in a sample from a human subject; and
b) identifying the human subject as being at risk for developing lung cancer based on the level of PSP-94 and the level of the at least one, two, three, four, five, or six proteins.

2. A method comprising:
a) measuring the level of PH protein and the level of at least one, two, three, four, five, or six proteins selected from the group consisting of MMP-12, SP-D, HE4, PSP-94, FUT5 and CRLF1 in a sample from a human subject; and
b) identifying the human subject as being at risk for developing lung cancer based on the level of PH and the level of the at least one, two, three, four, five, or six proteins.

3 A method comprising:
a) measuring the level of FUT5 protein and the level of at least one, two, three, four, five, or six proteins selected from the group consisting of MMP-12, SP-D, HE4, PSP-94, PH and CRLF1 in a sample from a human subject; and
b) identifying the human subject as being at risk for developing lung cancer based on the level of FUT5 and the level of the at least one, two, three, four, five, or six proteins.

4. A method comprising:
a) measuring the level of CRLF1 protein and the level of at least one, two, three, four, five, or six proteins selected from the group consisting of MMP-12, SP-D, HE4, PSP-94, PH and FUT5 in a sample from a human subject; and
b) identifying the human subject as being at risk for developing lung cancer based on the level of CRLF1 and the level of the at least one, two, three, four, five, or six proteins.

5. A method comprising:
a) contacting a sample from a human subject with a set of capture reagents, wherein each capture reagent has affinity for a different protein of the set of proteins comprising PSP-94 protein, and at least one, two, three, four, five, or six proteins selected from the group consisting of MMP-12, SP-D, HE4, PH, FUT5 and CRLF1; and
b) measuring the level of each protein of the set of proteins with the set of capture reagents.

6. A method comprising:
a) contacting a sample from a human subject with a set of capture reagents, wherein each capture reagent has affinity for a different protein of the set of proteins comprising PH protein, and at least one, two, three, four, five, or six proteins selected from the group consisting of MMP-12, SP-D, HE4, PSP-94, FUT5 and CRLF1; and
b) measuring the level of each protein of the set of proteins with the set of capture reagents.

7. A method comprising:
a) contacting a sample from a human subject with a set of capture reagents, wherein each capture reagent has affinity for a different protein of the set of proteins comprising FUT5 protein, and at least one, two, three, four, five, or six proteins selected from the group consisting of MMP-12, SP-D, HE4, PSP-94, PH and CRLF1; and
b) measuring the level of each protein of the set of proteins with the set of capture reagents.

8. A method comprising:
a) contacting a sample from a human subject with a set of capture reagents, wherein each capture reagent has affinity for a different protein of the set of proteins comprising CRLF1 protein, and at least one, two, three, four, five, or six proteins selected from the group consisting of MMP-12, SP-D, HE4, PSP-94, PH and FUT5; and
b) measuring the level of each protein of the set of proteins with the set of capture reagents.

9. The method of aspect 1 or aspect 5, wherein the method comprises measuring PSP-94 and MMP-12; PSP-94 and SP-D; PSP-94 and HE4; PSP-94 and PH; PSP-94 and FUT5; or PSP-94 and CRLF1.

10. The method of aspect 1 or aspect 5, wherein the method comprises measuring PSP-94, MMP-12 and SP-D; PSP-94, MMP-12 and HE4; PSP-94, MMP-12 and PH; PSP-94, MMP-12 and FUT5; PSP-94, MMP-12 and CRLF1; PSP-94, SP-D and HE4; PSP-94, SP-D and PH; PSP-94, SP-D and FUT5; PSP-94, SP-D and CRLF1; PSP-94, HE4 and PH; PSP-94, HE4 and FUT5; PSP-94, HE4 and CRLF1; PSP-94, PH and FUT5; PSP-94, PH and CRLF1; or PSP-94, FUT5 and CRLF1.

11. The method of aspect 2 or aspect 6, wherein the method comprises measuring PH and MMP-12; PH and SP-D; PH and HE4; PH and PSP-94; PH and FUT5; or PH and CRLF1.

12. The method of aspect 2 or aspect 6, wherein the method comprises measuring PH, MMP-12 and SP-D; PH, MMP-12 and HE4; PH, MMP-12 and PSP-94; PH, MMP-12 and FUT5; PH, MMP-12 and CRLF1; PH, SP-D and HE4; PH, SP-D and PSP-94; PH, SP-D and FUT5; PH, SP-D and CRLF1; PH, HE4 and PSP-94; PH, HE4 and FUT5; PH, HE4 and CRLF1; PH, PSP-94 and FUT5; PH, PSP-94 and CRLF1; or PH, FUT5 and CRLF1.

13. The method of aspect 3 or aspect 7, wherein the method comprises measuring FUT5 and MMP-12; FUT5 and SP-D; FUT5 and HE4; FUT5 and PSP-94; FUT5 and PH; or FUT5 and CRLF1.

14. The method of aspect 3 or aspect 7, wherein the method comprises measuring FUT5, MMP-12 and SP-D; FUT5, MMP-12 and HE4; FUT5, MMP-12 and PSP-94; FUT5, MMP-12 and PH; FUT5, MMP-12 and CRLF1; FUT5, SP-D and HE4; FUT5, SP-D and PSP-94; FUT5, SP-D and PH; FUT5, SP-D and CRLF1; FUT5, HE4 and PSP-94; FUT5, HE4 and PH; FUT5, HE4 and CRLF1; FUT5, PSP-94 and PH; FUT5, PSP-94 and CRLF1; or FUT5, PH and CRLF1.

15. The method of aspect 4 or aspect 8, wherein the method comprises measuring CRLF1 and MMP-12; CRLF1 and SP-D; CRLF1 and HE4; CRLF1 and PSP-94;CRLF1 and PH; or CRLF1 and FUT5.

16. The method of aspect 4 or aspect 8, wherein the method comprises measuring CRLF1, MMP-12 and SP-D; CRLF1, MMP-12 and HE4; CRLF1, MMP-12 and PSP-94; CRLF1, MMP-12 and PH; CRLF1, MMP-12 and FUT5; CRLF1, SP-D and HE4; CRLF1, SP-D and PSP-94; CRLF1, SP-D and PH; CRLF1, SP-D and FUT5; CRLF1, HE4 and PSP-94; CRLF1, HE4 and PH; CRLF1, HE4 and FUT5; CRLF1, PSP-94 and PH; CRLF1, PSP-94 and FUT5; or CRLF1, PH and FUT5.

17. The method of aspect 1 or aspect 5, wherein the method comprises measuring PSP-94 and PH and at least one of the following proteins selected from MMP-12, SP-D, HE4, FUT5 and CRLF1.

18. The method of aspect 1 or aspect 5, wherein the method comprises measuring PSP-94 and FUT5 and at least one of the following proteins selected from MMP-12, SP-D, HE4, PH and CRLF1.

19. The method of aspect 1 or aspect 5, wherein the method comprises measuring PSP-94 and CRLF1 and at least one of the following proteins selected from MMP-12, SP-D, HE4, PH and FUT5.

20. The method of aspect 2 or aspect 6, wherein the method comprises measuring PH and FUT5 and at least one of the following proteins selected from MMP-12, SP-D, HE4, PSP-94 and CRLF1.

21. The method of aspect 2 or aspect 6, wherein the method comprises measuring PH and CRLF1 and at least one of the following proteins selected from MMP-12, SP-D, HE4, PSP-94 and FUT5.

22. The method of aspect 3 or aspect 7, wherein the method comprises measuring FUT5 and CRLF1 and at least one of the following proteins selected from MMP-12, SP-D, HE4, PSP-94 and PH.

23. A method comprising:
a) contacting a sample from a human subject with two capture reagents, wherein one capture reagent has affinity for a PSP-94 protein and the second capture reagent has affinity for a PH protein; and
b) measuring the level of each protein with the two capture reagents.

24. A method comprising:
a) contacting a sample from a human subject with two capture reagents, wherein one capture reagent has affinity for a PSP-94 protein and the second capture reagent has affinity for a FUT5 protein; and
b) measuring the level of each protein with the two capture reagents.

25. A method comprising:
a) contacting a sample from a human subject with two capture reagents, wherein one capture reagent has affinity for a PSP-94 protein and the second capture reagent has affinity for a CRLF1 protein; and
b) measuring the level of each protein with the two capture reagents.

26. A method comprising:
a) contacting a sample from a human subject with two capture reagents, wherein one capture reagent has affinity for a PH protein and the second capture reagent has affinity for a FUT5 protein; and
b) measuring the level of each protein with the two capture reagents.

27. A method comprising:
a) contacting a sample from a human subject with two capture reagents, wherein one capture reagent has affinity for a PH protein and the second capture reagent has affinity for a CRLF1 protein; and
b) measuring the level of each protein with the two capture reagents.

28. A method comprising:
a) contacting a sample from a human subject with two capture reagents, wherein one capture reagent has affinity for a FUT5 protein and the second capture reagent has affinity for a CRLF1 protein; and
b) measuring the level of each protein with the two capture reagents.

29. A method comprising:
a) measuring the level of PSP-94 and PH in a sample from a human subject; and
b) identifying the human subject as being at risk for developing lung cancer based on the level of PSP-94 and PH.

30. A method comprising:
a) measuring the level of PSP-94 and FUT5 in a sample from a human subject; and
b) identifying the human subject as being at risk for developing lung cancer based on the level of PSP-94 and FUT5.

31. A method comprising:
a) measuring the level of PSP-94 and CRLF1 in a sample from a human subject; and
b) identifying the human subject as being at risk for developing lung cancer based on the level of PSP-94 and CRLF1.

32. A method comprising:
a) measuring the level of PH and FUT5 in a sample from a human subject; and
b) identifying the human subject as being at risk for developing lung cancer based on the level of PH and FUT5.

33. A method comprising:
a) measuring the level of PH and CRLF1 in a sample from a human subject; and
b) identifying the human subject as being at risk for developing lung cancer based on the level of PH and CRLF1.

34. A method comprising:
a) measuring the level of FUT5 and CRLF1 in a sample from a human subject; and
b) identifying the human subject as being at risk for developing lung cancer based on the level of FUT5 and CRLF1.

35. A method comprising:
a) contacting a sample from a human subject with three capture reagents, wherein each of the three capture reagents has affinity for a protein selected from PSP-94, PH, and FUT5; and
b) measuring the level of each protein with the three capture reagents.

36. A method comprising:
a) contacting a sample from a human subject with three capture reagents, wherein each of the three capture reagents has affinity for a protein selected from PSP-94, PH, and CRLF1; and
b) measuring the level of each protein with the three capture reagents.

37. A method comprising:
a) contacting a sample from a human subject with three capture reagents, wherein each of the three capture reagents has affinity for a protein selected from PH, FUT5, and CRLF1; and
b) measuring the level of each protein with the three capture reagents.

38. A method comprising:
a) contacting a sample from a human subject with three capture reagents, wherein each of the three capture reagents has affinity for a protein selected from FUT5, CRLF1, and PSP-94; and
b) measuring the level of each protein with the three capture reagents.

39. A method comprising:
a) measuring the level of PSP-94, PH, and FUT5 in a sample from a human subject; and
b) identifying the human subject as being at risk for developing lung cancer based on the level of PSP-94, PH, and FUT5.

40. A method comprising:
a) measuring the level of PSP-94, PH, and CRLF1 in a sample from a human subject; and
b) identifying the human subject as being at risk for developing lung cancer based on the level of PSP-94, PH, and CRLF1.

41. A method comprising:
a) measuring the level of PH, FUT5, and CRLF1 in a sample from a human subject; and
b) identifying the human subject as being at risk for developing lung cancer based on the level of PH, FUT5, and CRLF1.

42. A method comprising:
a) measuring the level of FUT5; CRLF1, and PSP-94 in a sample from a human subject; and
b) identifying the human subject as being at risk for developing lung cancer based on the level of FUT5; CRLF1, and PSP-94.

43. The method of any one of aspects 23-42, further comprising measuring the level of MMP-12 protein.

44. The method of any one of aspects 23-43, further comprising measuring the level of SP-D protein.

45. The method of any one of aspects 23-44, further comprising measuring the level of HE4 protein.

46. A method comprising:
a) measuring the level of at least three, four, five, six, or seven proteins selected from the group consisting of MMP-12, SP-D, HE4, PSP-94, PH, FUT5 and CRLF1 in a sample from a human subject; and
b) identifying the human subject as being at risk for developing lung cancer based on the level of the at least three, four, five, six, or seven proteins.

47. The method of aspect 46, wherein the method comprises measuring MMP-12, SP-D and HE4; MMP-12, SP-D and PSP-94; MMP-12, SP-D and PH; MMP-12, SP-D and FUT5; MMP-12, SP-D and CRLF1; MMP-12, HE4 and PSP-94; MMP-12, HE4 and PH; MMP-12, HE4 and FUT5; MP-12, HE4 and CRLF1; MMP-12, PSP-94 and PH; MMP-12, PSP-94 and FUT5; MMP-12, PSP-94 and CRLF1; MMP-12, PH and FUT5; MMP-12, PH and CRLF1; MMP-12, FUT5 and CRLF1; SP-D, HE4 and PSP-94; SP-D, HE4 and PH; SP-D, HE4 and FUT5; SP-D, HE4 and CRLF1; SP-D, PSP-94 and PH; SP-D, PSP-94 and FUT5; SP-D, PSP-94 and CRLF1; SP-D, PH and FUT5; SP-D, PH and CRLF1; SP-D, FUT5 and CRLF1; HE4, PSP-94 and PH; HE4, PSP-94 and FUT5; HE4, PSP-94 and CRLF1; HE4, PH and FUT5; HE4, PH and CRLF1; HE4, FUT5 and CRLF1; PSP-94, PH and FUT5; PSP-94, PH and CRLF1; PSP-94, FUT5 and CRLF1; or PH, FUT5, and CRLF1.

48. The method of aspect 46 or 47, further comprising measuring one or more of PSP-94, PH, FUT5, and CRLF1.

49. A method comprising:
a) contacting a sample from a human subject with a set of capture reagents, wherein each capture reagent has affinity for a different protein of the set of proteins comprising at least three, four, five, six, or seven proteins selected from the group consisting of MMP-12, SP-D, HE4,PSP-94, PH, FUT5 and CRLF1 in a sample from a human subject; and b) measuring the level of each protein of the set of proteins with the set of capture reagents.

50. The method of aspect 49, wherein the method comprises measuring MMP-12, SP-D and HE4; MMP-12, SP-D and PSP-94; MMP-12, SP-D and PH; MMP-12, SP-D and FUT5; MMP-12, SP-D and CRLF1; MMP-12, HE4 and PSP-94; MMP-12, HE4 and PH; MMP-12, HE4 and FUT5; MP-12, HE4 and CRLF1; MMP-12, PSP-94 and PH; MMP-12, PSP-94 and FUT5; MMP-12, PSP-94 and CRLF1; MMP-12, PH and FUT5; MMP-12, PH and CRLF1; MMP-12, FUT5 and CRLF1; SP-D, HE4 and PSP-94; SP-D, HE4 and PH; SP-D, HE4 and FUT5; SP-D, HE4 and CRLF1; SP-D, PSP-94 and PH; SP-D, PSP-94 and FUT5; SP-D, PSP-94 and CRLF1; SP-D, PH and FUT5; SP-D, PH and CRLF1; SP-D, FUT5 and CRLF1; HE4, PSP-94 and PH; HE4, PSP-94 and FUT5; HE4, PSP-94 and CRLF1; HE4, PH and FUT5; HE4, PH and CRLF1; HE4, FUT5 and CRLF1; PSP-94, PH and FUT5; PSP-94, PH and CRLF1; PSP-94, FUT5 and CRLF1; or PH, FUT5, and CRLF1.

51. The method of aspect 49 or 50, further comprising measuring one or more of PSP-94, PH, FUT5 and CRLF1.

52. A method comprising:
a) measuring the level of MMP-12 protein, and the level of at least one, two, three, four, five, or six proteins selected from the group consisting of SP-D, HE4, PSP-94, PH, FUT5 and CRLF1 in a sample from a human subject; and
b) identifying the human subject as being at risk for developing lung cancer based on the level of MMP-12 and the level of the at least one, two, three, four, five, or six, proteins.

53. A method comprising:
a) measuring the level of SP-D protein, and the level of at least one, two, three, four, five, or six proteins selected from the group consisting of MMP-12, HE4, PSP-94, PH, FUT5 and CRLF1 in a sample from a human subject; and
b) identifying the human subject as being at risk for developing lung cancer based on the level of SP-D and the level of the at least one, two, three, four, five, six, seven, eight, or nine proteins.

54. A method comprising:
a) measuring the level of HE4 protein, and the level of at least one, two, three, four, five, or six proteins selected from the group consisting of MMP-12, SP-D, PSP-94, PH, FUT5 and CRLF1 in a sample from a human subject; and
b) identifying the human subject as being at risk for developing lung cancer based on the level of HE4 and the level of the at least one, two, three, four, five, six, seven, eight, or nine proteins.

55. A method comprising:
a) contacting a sample from a human subject with a set of capture reagents, wherein each capture reagent has affinity for a different protein of the set of proteins comprising MMP-12 protein, and at least one, two, three, four, five, or six proteins selected from the group consisting of SP-D, HE4, PSP-94, PH, FUT5 and CRLF1; and
b) measuring the level of each protein of the set of proteins with the set of capture reagents.

56. A method comprising:
a) contacting a sample from a human subject with a set of capture reagents, wherein each capture reagent has affinity for a different protein of the set of proteins comprising SP-D protein, and at least one, two, three, four, five, or six proteins selected from the group consisting of MMP-12, HE4, PSP-94, PH, FUT5 and CRLF1; and
b) measuring the level of each protein of the set of proteins with the set of capture reagents. 57. A method comprising:
a) contacting a sample from a human subject with a set of capture reagents, wherein each capture reagent has affinity for a different protein of the set of proteins comprising HE4 protein, and at least one, two, three, four, five, or six proteins selected from the group consisting of MMP-12, SP-D, PSP-94, PH, FUT5 and CRLF1; and
b) measuring the level of each protein of the set of proteins with the set of capture reagents.

58. The method of any one of aspects 5-28, 36-38, 49-51, and 55-57, wherein the set of capture reagents is selected from aptamers, antibodies and a combination of aptamers and antibodies.

59. The method of any one of the preceding aspects, wherein the measuring is performed using mass spectrometry, an aptamer based assay and/or an antibody based assay.

60. The method of aspect 58 or claim 59, wherein the level of each biomarker protein measured is determined from a relative florescence unit (RFU) or a protein concentration.

61. The method of any one of the preceding aspects, wherein the sample is selected from blood, plasma, serum or urine.

62. The method of any one of the preceding aspects, wherein the protein levels are used to identify a human subject as being at risk for developing lung cancer.

63. The method of aspect 62, wherein the risk for developing lung cancer is within a 5 year period.

64. The method of aspect 62 or aspect 63, wherein the risk for developing lung cancer is within a period of 1 year, 2 years, 3 years, 4 years, 5 years, 6 years, 7 years, 8 years, 9 years, or 10 years.

65. The method of any one of the preceding aspects, wherein the human subject is a current smoker or a former smoker.

66. The method of any one of the preceding aspects, wherein the subject has lung

cancer.

67. The method of any one of the preceding aspects, wherein the method provides an area under the curve (AUC) of 0.62, 0.67, 0.68, 0.68, 0.70, 0.71, 0.72, 0.73, 0.74, 0.75, 0.76, or above.

68. The method of any one of the preceding aspects, wherein the method provides an area under the curve (AUC) from about 0.6 to about 0.8, from about 0.61 to about 0.78, from about 0.62 to about 0.76, from about 0.62 to about 0.68, from about 0.67 to about 0.72, from about 0.69 to about 0.74, from about 0.71 to about 0.74, from about 0.73 to about 0.76, or from about 0.74 to about 0.76.

69. The method of any one of the preceding aspects, wherein predicting the risk for developing lung cancer is based on input of the levels of the measured proteins in a statistical model.

70. The method of aspect 69, wherein the predicting comprises analyzing the levels of the measured proteins using an Accelerated Failure Time (AFT) Weibull survival model.

71. The method of any one of the preceding aspects, further comprising

performing a diagnostic screening.

72. The method of aspect 71, wherein the diagnostic screening is selected from low dose computed tomography (LDCT), chest radiography, and sputum cytology.

73. The method of any one of the preceding aspects, wherein the method comprises predicting the risk for developing lung cancer for the purpose of determining a medical insurance premium or life insurance premium.

74. The method of aspect 73, wherein the method further comprises determining coverage or premium for medical insurance or life insurance.

75. The method of any one of aspects 1-72, wherein the method further comprises using information resulting from the method to predict and/or manage the utilization of medical resources.

76. The method of any one of aspects 1-72, wherein the method further comprises using information resulting from the method to enable a decision to acquire or purchase a medical practice, hospital, or company.

77. A kit comprising N protein capture reagents, wherein N is at least 1, at least 2, at least 3, at least 4, at least 5, at least 6, or at least 7, and wherein at least one of the N protein capture reagents specifically binds to a protein selected from PSP-94, MMP-12, SP-D, HE4, PH, FUT5 and CRLF1.

78. The kit of aspect 77, wherein N is at least two and at least one to the two N protein capture reagents specifically binds to the protein selected from PSP-94, MMP-12, SP-D, HE4, PH, FUT5 and CRLF1.

79. The kit of aspect 77 or 78, wherein N is 2 to 7, or Nis 3 to 7, or Nis 4 to 7, or Nis 5 to 7, or Nis 6 to 7.

80. The kit of any one of aspects 77-79, wherein Nis 2, Nis 3, Nis 4, Nis 5, N is 6, or Nis 7.

81. The kit of any one of aspects 77-80, wherein each of the N protein capture reagents specifically binds to a different biomarker protein.

82. The kit of any one of aspects 77-81, wherein each of the N protein capture reagents specifically binds to a protein selected from PSP-94, MMP-12, SP-D, HE4, PH, FUT5 and CRLF1.

83. The kit of any one of aspects 77-81, wherein two of the N protein capture reagents specifically bind PSP-94 and MMP-12; or two of the N protein capture reagents specifically bind PSP-94 and SP-D; or two of the N protein capture reagents specifically bind PSP-94 and HE4; or two of the N protein capture reagents specifically bind PSP-94 and PH; or two of the N protein capture reagents specifically bind PSP-94 and FUT5; or two of the N protein capture reagents specifically bind PSP-94 and CRLF1.

84. The kit of any one of aspects 77-81, wherein three of the N protein capture reagents specifically bind PSP-94, MMP-12 and SP-D; or three of the N protein capture reagents specifically bind PSP-94, MMP-12 and HE4; or three of the N protein capture reagents specifically bind PSP-94, MMP-12 and PH; or three of the N protein capture reagents specifically bind PSP-94, MMP-12 and FUT5; or three of the N protein capture reagents specifically bind PSP-94, MMP-12 and CRLF1; or three of the N protein capture reagents specifically bind PSP-94, SP-D and HE4; or three of the N protein capture reagents specifically bind PSP-94, SP-D and PH; or three of the N protein capture reagents specifically bind PSP-94, SP-D and FUT5; or three of the N protein capture reagents specifically bind PSP-94, SP-D and CRLF1; or three of the N protein capture reagents specifically bind PSP-94, HE4 and PH; PSP-94, HE4 and FUT5; or three of the N protein capture reagents specifically bind PSP-94, HE4 and CRLF1; or three of the N protein capture reagents specifically bind PSP-94, PH and FUT5; or three of the N protein capture reagents specifically bind PSP-94, PH and CRLF1; or three of the N protein capture reagents specifically bind PSP-94, FUT5 and CRLF1.

85. The kit of any one of aspects 77-81, wherein two of the N protein capture reagents specifically bind PH and MMP-12; or two of the N protein capture reagents specifically bind PH and SP-D; or two of the N protein capture reagents specifically bind PH and HE4; or two of the N protein capture reagents specifically bind PH and PSP-94; PH and FUT5; or two of the N protein capture reagents specifically bind PH and CRLF1.

86. The kit of any one of aspects 77-81, wherein three of the N protein capture reagents specifically bind PH, MMP-12 and SP-D; or three of the N protein capture reagents specifically bind PH, MMP-12 and HE4; or three of the N protein capture reagents specifically bind PH, MMP-12 and PSP-94; or three of the N protein capture reagents specifically bind PH, MMP-12 and FUT5; or three of the N protein capture reagents specifically bind PH, MMP-12 and CRLF1; or three of the N protein capture reagents specifically bind PH, SP-D and HE4; or three of the N protein capture reagents specifically bind PH, SP-D and PSP-94; or three of the N protein capture reagents specifically bind PH, SP-D and FUT5; or three of the N protein capture reagents specifically bind PH, SP-D and CRLF1; or three of the N protein capture reagents specifically bind PH, HE4 and PSP-94; or three of the N protein capture reagents specifically bind PH, HE4 and FUT5; or three of the N protein capture reagents specifically bind PH, HE4 and CRLF1; or three of the N protein capture reagents specifically bind PH, PSP-94 and FUT5; PH, PSP-94 and CRLF1; or three of the N protein capture reagents specifically bind PH, FUT5 and CRLF1.

87. The kit of any one of aspects 77-81, wherein two of the N protein capture reagents specifically bind FUT5 and MMP-12; or two of the N protein capture reagents specifically bind FUT5 and SP-D; or two of the N protein capture reagents specifically bind FUT5 and HE4; or two of the N protein capture reagents specifically bind FUT5 and PSP-94; or two of the N protein capture reagents specifically bind FUT5 and PH; or two of the N protein capture reagents specifically bind FUT5 and CRLF1.

88. The kit of any one of aspects 77-81, wherein three of the N protein capture reagents specifically bind FUT5, MMP-12 and SP-D; or three of the N protein capture reagents specifically bind FUT5, MMP-12 and HE4; or three of the N protein capture reagents specifically bind FUT5, MMP-12 and PSP-94; or three of the N protein capture reagents specifically bind FUT5, MMP-12 and PH; or three of the N protein capture reagents specifically bind FUT5, MMP-12 and CRLF1; or three of the N protein capture reagents specifically bind FUT5, SP-D and HE4; or three of the N protein capture reagents specifically bind FUT5, SP-D and PSP-94; or three of the N protein capture reagents specifically bind FUT5, SP-D and PH; or three of the N protein capture reagents specifically bind FUT5, SP-D and CRLF1; or three of the N protein capture reagents specifically bind FUT5, HE4 and PSP-94; or three of the N protein capture reagents specifically bind FUT5, HE4 and PH; or three of the N protein capture reagents specifically bind FUT5, HE4 and CRLF1; or three of the N protein capture reagents specifically bind FUT5, PSP-94 and PH; or three of the N protein capture reagents specifically bind FUT5, PSP-94 and CRLF1; or three of the N protein capture reagents specifically bind FUT5, PH and CRLF1.

89. The kit of any one of aspects 77-81, wherein two of the N protein capture reagents specifically bind CRLF1 and MMP-12; or two of the N protein capture reagents specifically bind CRLF1 and SP-D; or two of the N protein capture reagents specifically bind

CRLF1 and HE4; or CRLF1 and PSP-94; or two of the N protein capture reagents specifically bind CRLF1 and PH; or two of the N protein capture reagents specifically bind CRLF1 and FUT5.

90. The kit of any one of aspects 77-81, wherein three of the N protein capture reagents specifically bind CRLF1, MMP-12 and SP-D; or three of the N protein capture reagents specifically bind CRLF1, MMP-12 and HE4; or three of the N protein capture reagents specifically bind CRLF1, MMP-12 and PSP-94; or three of the N protein capture reagents specifically bind CRLF1, MMP-12 and PH; or three of the N protein capture reagents specifically bind CRLF1, MMP-12 and FUT5; or three of the N protein capture reagents specifically bind CRLF1, SP-D and HE4; or three of the N protein capture reagents specifically bind CRLF1, SP-D and PSP-94; or three of the N protein capture reagents specifically bind CRLF1, SP-D and PH; or three of the N protein capture reagents specifically bind CRLF1, SP-D and FUT5; or three of the N protein capture reagents specifically bind CRLF1, HE4 and PSP-94; or three of the N protein capture reagents specifically bind CRLF1, HE4 and PH; or three of the N protein capture reagents specifically bind CRLF1, HE4 and FUT5; or three of the N protein capture reagents specifically bind CRLF1, PSP-94 and PH; or three of the N protein capture reagents specifically bind CRLF1, PSP-94 and FUT5; or three of the N protein capture reagents specifically bind CRLF1, PH and FUT5.

91. A kit comprising N protein capture reagents, wherein the kit comprises protein capture reagents for carrying out the methods any one of claims 1-76.

92. The kit of any one of aspects 77-91, wherein each of the N biomarker protein capture reagents is an antibody or an aptamer.

93. The kit of aspect 92, wherein each biomarker protein capture reagent is an aptamer.

94. The kit of any one of aspects 77-93, for use in detecting the N biomarker proteins in a sample from a subject.

95. The kit of aspect 94, for use in predicting risk of a subject for developing lung cancer. 96. The kit of aspect 95, wherein the subject has lung cancer.

### BRIEF DESCRIPTION OF THE DRAWINGS

FIG. 1 shows the effect of smoking, and smoking cessation on lifetime risk for lung cancer in men and women since 1995. Image from Bruder et al. “Estimating lifetime and 10-year risk of lung cancer.” Prev Med Rep 2018; 11:125-30.

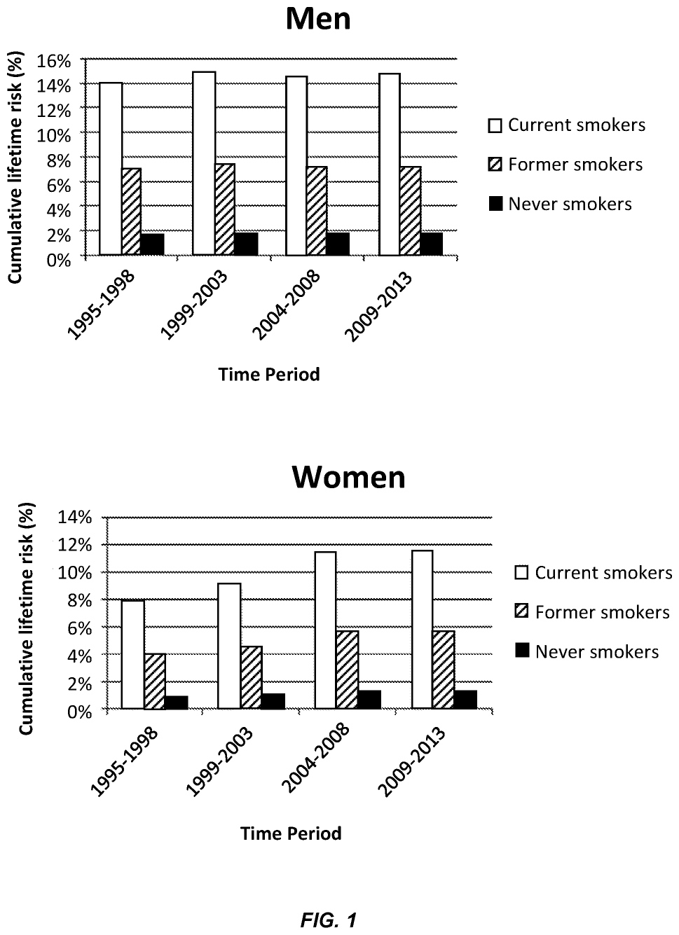

FIG. 2 shows Kaplan-Meier plots of training and verification data using relative risk bins.

FIG. 3 illustrates an exemplary computer system for use with various computer-implemented methods described herein.

FIG. 4 is a flowchart for a method of evaluating risk of lung cancer in accordance with one embodiment.

FIG. 5 shows a line plot of lung cancer risk predictions from visit 2 to visit 3 stratified by an individual's smoking behavior changes across time.

FIG. 6 shows a boxplot of lung cancer predictions in individuals with prevalent lung cancer (Y) and individuals who do not (N) at ARIC visit 3.

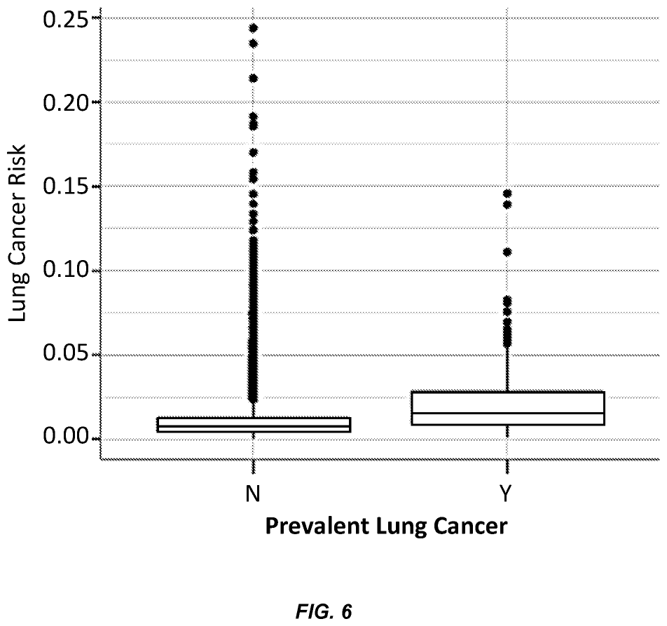

FIG. 7 shows a boxplot of lung cancer predictions in individuals with prevalent lung cancer (Y) and individuals who do not (N) at ARIC visit 5.

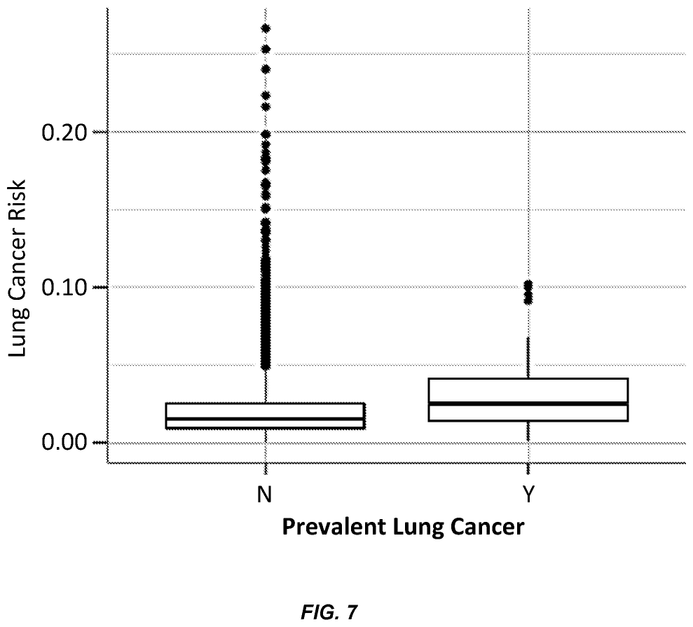

### DETAILED DESCRIPTION

Reference will now be made in detail to representative embodiments of the

invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.” Thus, reference to “a SOMAmer” includes mixtures of SOMAmers, reference to “a probe” includes mixtures of probes, and the like.

As used herein, the term “about” represents an insignificant modification or variation of the numerical value such that the basic function of the item to which the numerical value relates is unchanged.

As used herein, “backward selection” refers to a method for feature selection and reduction. In certain aspects, backward selection is a form of stepwise regression that starts with all features included in a model. For example, in an iterative process, features are considered for subtraction using AUC as the selection criterion.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), dried blood spots (e.g., obtained from infants), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, it should be realized that a biological sample can be derived by taking biological samples from a number of individuals and pooling them or pooling an aliquot of each individual's biological sample.

As mentioned above, the biological sample can be urine. Urine samples provide certain advantages over blood or serum samples. Collecting blood or plasma samples through venipuncture is more complex than is desirable, can deliver variable volumes, can be worrisome for the patient, and involves some (small) risk of infection. Also, phlebotomy requires skilled personnel. The simplicity of collecting urine samples can lead to more widespread application of the subject methods.

“Computation” as used herein refers to any type of mathematical calculation, including arithmetical and non-arithmetical steps.

For purposes of this specification, the phrase “data attributed to a biological sample from an individual” is intended to mean that the data in some form derived from, or were generated using, the biological sample of the individual. The data may have been reformatted, revised, or mathematically altered to some degree after having been generated, such as by conversion from units in one measurement system to units in another measurement system; but, the data are understood to have been derived from, or were generated using, the biological sample.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” is a set of copies of one type or species of molecule or multi-molecular structure. “Target molecules”, “targets”, and “analytes” refer to more than one such set of molecules. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In certain aspects, “analyte” is the protein target of a capture reagent, e.g. an aptamer. In certain further aspects, the capture reagent is a SOMAmer.

As used herein, “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be single chains or associated chains. Also included within the definition are preproteins and intact mature proteins; peptides or polypeptides derived from a mature protein; fragments of a protein; splice variants; recombinant forms of a protein; protein variants with amino acid modifications, deletions, or substitutions; digests; and post-translational modifications, such as glycosylation, acetylation, phosphorylation, and the like.

As used herein, “marker” and “biomarker” and “feature” are used interchangeably to refer to a target molecule that indicates or is a sign of a normal or abnormal process in an individual or of a disease or other condition in an individual. More specifically, a “marker” or “biomarker” or “feature” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker. In certain aspects, a feature is an analyte/SOMAmer reagent of other predictors in a statistical model.

As used herein, “biomarker value”, “value”, “biomarker level”, “feature level” and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker is generally described as being either over-expressed or under-expressed as compared to an expression level or value of the biomarker that indicates or is a sign of a normal process or an absence of a disease or other condition in an individual. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease or condition, relative to its expression in a normal or control subject. The terms also include genes (or the corresponding protein expression products) whose expression is activated to a higher or lower level at different stages of the same disease or condition. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, “individual” refers to a test subject or patient. The individual can be a mammal or a non-mammal. In various embodiments, the individual is a mammal. A mammalian individual can be a human or non-human. In various embodiments, the individual is a human. A healthy or normal individual is an individual in which the disease or condition of interest (including, for example, lung cancer) is not detectable by conventional diagnostic methods.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual.

As used herein, “elastic net logistic regression” refers to a machine learning method that utilizes penalized regression techniques to select the features that best predict the endpoint while allowing correlated features to be grouped together.

As used herein, “feature” refers to an analyte or other predictor in a statistical model.

As used herein, “forward selection” refers to a method for feature selection and reduction. In certain aspects, it is a form of stepwise regression that starts with zero features included in the model. For example, in an iterative process, features are considered for addition using AUC as the selection criterion.

As used herein “Mean Absolute Error” or “MAE” refers to the mean of the absolute values of the prediction error on all instances of a dataset.

As used herein, “Normalized Root Mean Square Error or “NRMSE” refers to the standard deviation of prediction errors (residuals) divided by the mean of the outcome. As used herein, “parent study” refers to an external study source for a test study.

As used herein, “prediction error curve” or “Brier score” refers to the difference between the predicted survival time vs. the observed survival time for each individual, thus higher values represent a worse model.

As used herein, “population adaptive median normalization” refers to a process for normalizing the analytes to mitigate site-bias and sample-handing issues.

As used herein, “principle component analysis” refers to a method for assessing and identifying large sources of variation in the data.

As used herein, “Root Mean Square Error” or “RMSE” refers to the standard deviation of prediction errors (residuals).

As used herein the term “predict” refers to an estimation regarding a state or a condition in the present or in the future. In one aspect, to predict or making a prediction refers to an estimation regarding the risk of lung cancer within a specified time period. In one aspect, the time period is 5 years. In one aspect, the subject has lung cancer.

“Prognose”, “prognosing”, “prognosis”, and variations thereof refer to the prediction of a future course of a disease or condition in an individual who has the disease or condition (e.g., predicting patient survival), and such terms encompass the evaluation of disease or condition response after the administration of a treatment or therapy to the individual.

As used herein the term “R²” refers to the proportion of the variance in outcome that can be explained by a model.

“Evaluate”, “evaluating”, “evaluation”, and variations thereof encompass both “diagnose” and “prognose” and also encompass determinations or estimations about the current or future course of a disease or condition in an individual who may or may not have the disease as well as determinations or estimations regarding the risk that a disease or condition will recur in an individual who apparently has been cured of the disease or has had the condition resolved. The term “evaluate” also encompasses assessing an individual's response to a therapy, such as, for example, determining whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual.”

As used herein, “additional biomedical information” refers to one or more evaluations of an individual, other than using any of the biomarkers described herein, that are associated current state of lung health. “Additional biomedical information” includes any of the following: physical descriptors of an individual, including the height and/or weight of an individual; the age of an individual; the gender of an individual; change in weight; the ethnicity of an individual; occupational history; family history of lung cancer; the presence of a genetic marker(s) correlating with a higher risk of lung cancer in the individual; clinical symptoms such as chest pain, weight gain or loss gene expression values; physical descriptors of an individual, including physical descriptors observed by radiologic imaging; smoking status; alcohol use history; occupational history; dietary habits-salt, saturated fat and cholesterol intake; caffeine consumption; and imaging information. Testing of biomarker levels in combination with an evaluation of any additional biomedical information, including other laboratory tests, may, for example, improve sensitivity, specificity, and/or AUC for estimation or determination of current state of lung health as compared to biomarker testing alone or evaluating any particular item of additional biomedical information alone. Additional biomedical information can be obtained from an individual using routine techniques known in the art, such as from the individual themselves by use of a routine patient questionnaire or health history questionnaire, etc., or from a medical practitioner, etc. Testing of biomarker levels in combination with an evaluation of any additional biomedical information may, for example, improve sensitivity, specificity, and/or thresholds for estimation or determination of the current state of lung health as compared to biomarker testing alone or evaluating any particular item of additional biomedical information alone (e.g., CT imaging alone).

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore-or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly) tetrafluoroethylene, (poly) vinylidenefluoride, polycarbonate, and polymethylpentene. Suitable solid support particles that can be used include, e.g., encoded particles, such as Luminex®-type encoded particles, magnetic particles, and glass particles.

As used herein, “stability selection” refers to a method for feature selection and reduction that uses regularization techniques and subsampling approaches such that the Type I error rate is controlled throughout the feature selection process.

As used herein, “adaptive normalization by maximum likelihood” means a process for normalizing the analytes to mitigate site bias.

As used herein, “Lin's Concordance correlation coefficient” or “Lin's CCC” means concordance correlation coefficient which measures the concordance between a new test and an existing test that is considered the gold standard.

As used herein, “study”, means a set of samples and clinical data that are analyzed to derive the test.

As used herein, “test dataset”, means a final subset of data used to assess the performance of the final model developed on the verification dataset.

As used herein, “training dataset”, means a subset of data from a study used to fit a model.

As used herein, “validation dataset”, means a final subset of data used to assess the performance of a final model developed on a verification dataset.

As used herein, “verification dataset”, means a separate subset of data used to provide an unbiased evaluation of a model fit on the training dataset while tuning model parameters.

As used herein, the term “need” or “needed” refers to a judgement made by a health care provider regarding treatment of a patient which is considered by the health care provider to be beneficial to the health status of the patient.

In one aspect, a lung cancer risk test is disclosed providing a model that predicts a current or former smoker's risk for a lung cancer diagnosis within a specified period of time, for example within 5 years of blood draw.

In certain aspects, the endpoint used for model development is a lung cancer diagnosis adjudicated by electronic health record and cancer registry review.

In one aspect, a lung cancer risk test was developed using the Atherosclerosis Risk in Communities (ARIC) visit 3 cohort which was split into training (70%), verification (15%), and validation (15%) datasets. The ARIC study was initially intended to longitudinally investigate the contributions of genetic, environmental, and demographic risk factors to atherosclerosis and related cardiovascular diseases; however, the study objectives expanded to also investigate cancer-related outcomes. (Joshu et al. “Enhancing the Infrastructure of the Atherosclerosis Risk in Communities (ARIC) Study for Cancer Epidemiology Research: ARIC Cancer.” Cancer Epidemiol Biomarkers Prev 2018;27:295-305).

In certain aspects, the intended use population for this test is adults, aged 50 years or older, who are current or former smokers and eligible for lung cancer screening under current guidelines. The final model is a 7-feature, protein-only, accelerated failure time (AFT) Weibull model. The model output may be reported as the absolute risk probability of a lung cancer diagnosis within 5 years, or a relative risk probability of a lung cancer diagnosis within 5 years, as compared to the average risk in the “ever smoker” cohort used for model development. The range of relative risk is 0.010-25.

In one aspect, the minimum performance requirement for this test was an area under the curve (AUC) at least equivalent to the published performance of the national Lung Cancer Screening Trial (NLST) to predict future lung cancer risk in current and former smokers (AUC=0.689) (Tammemagi, et al. “Selection criteria for lung-cancer screening.” N Engl J Med 2013;368:728-36). Training and verification results exceed the performance metric of an AUC≥0.689 (Table 1). Additional exploratory analysis was also performed to assess the lung cancer risk model performance in never smokers.

TABLE 1
Performance of final lung cancer risk model at 5 years on training, verification, and validation data.
Training, verification, and validation results exceed performance criteria of AUC ≥0.689.

| | | AUC | Sensitivity | Specificity | | PEC |
|---|---|---|---|---|---|---|
| | N per | at 5 years | at 5 years | at 5 years | C-Index* | at 5 years |
| Dataset | group | (95% CI) | (95% CI) | (95% CI) | (95% CI) | (95% CI) |
| Training | 4260 | 0.76 | 0.70 | 0.72 | 0.73 | 0.01 |
| | | (0.69-0.82) | (0.51-0.90) | (0.51-0.87) | (0.70-0.76) | (0.009-0.02) |
| Verification | 913 | 0.72 | 0.88 | 0.59 | 0.71 | 0.01 |
| | | (0.55-0.89) | (0.43-1) | (0.54-0.95) | (0.62-0.79) | (0.005-0.02) |
| Validation | 912 | 0.83 | 0.76 | 0.73 | 0.74 | 0.02 |
| | | (0.75-0.91) | (0.67-1) | (0.43-0.90) | (0.67-0.78) | (0.01-0.03) |

AUC, Area Under the Curve; PEC, Prediction Error Curve; CI, Confidence Intervals.
*C-Index is not time specific.

In certain aspects, the intended use of the lung cancer risk test disclosed herein is to predict an individual's risk probability of a lung cancer diagnosis within 5 years of the blood sample. In further aspects, the test is intended for cancer-free adults aged 50 and above who are current or former smokers and eligible for lung cancer screening under current guidelines. In certain aspects, the test is not intended for use in individuals with current known cancer. For RUO use, the benefits and risks pertain to decision making in research studies for participant monitoring, stratification, and enrichment. The benefit/risk analysis for clinical LDT use is described below.

Benefits of the presently disclosed lung cancer risk test include: a convenient way to gain personalized knowledge of the degree of risk for a future lung cancer diagnosis without reliance on self-reported demographics or genetic background information; the test result may influence compliance with lung cancer screening guidelines allowing for the potential for earlier identification of lung cancer, and thus improved chance of lung cancer survival; the test may influence positive behavior changes in modifiable risk-related behaviors (e.g., smoking cessation, dietary change, weight loss); and the test may aid healthcare provider lung cancer screening decisions/recommendations or patient's lung screening preferences based on the test result (e.g., for patients in high-risk category to initially undertake LDCT screening methodology that is considered the gold standard versus another lesser sensitive methodology such as chest radiography).

The test disclosed herein can be used in conjunction with additional assessments including but not limited to health status assessments, including evaluations of comorbid conditions such as diabetes, additional laboratory tests including but not limited to measurement of serum creatinine, urine albumin, clinical pathology, lung imaging, and histology.

In one aspect, one or more biomarkers are provided for use either alone or in various combinations to predict lung cancer risk. As described in detail below, exemplary embodiments include the biomarkers provided in Table 6, which were identified using a multiplex SOMAmer-based assay.

In a preferred embodiment, the model has 7 features (Table 6) for prediction of

lung cancer risk.

In one embodiment, the number of biomarkers useful for a biomarker subset or

panel is based on a selection of biomarkers with non-zero coefficients as a measure of prediction power for lung cancer risk.

Risk analysis is provided in Table 2.

TABLE 2
Risk Analysis

| | | | | Need for |
|---|---|---|---|---|
| | | Likelihood | | Mitigating |
| Hazard | Hazardous Situation(s) | relative to SoC | Harm(s) | Measures |
| False Positive | HCP recommends lung | [ ] Lower | No harm as patient is | [✓] Low |
| Result | cancer screening | [✓] Equivalent | already in screening | [ ] Med |
| (erroneous | | [ ] Higher | eligible population and | [ ] High |
| overprediction | | | current SoC guidelines | |
| of lung cancer | | | recommend annual lung | |
| risk) | | | cancer screening | |
| | HCP recommends lung | | Potential for discomfort | |
| | cancer screening with | | and inconvenience for | |
| | higher level of | | patient. | |
| | invasiveness (e.g. LDCT | | Minimal increase in risk | |
| | vs sputum cytology) | | for screening-related | |
| | | | complications (e.g. level | |
| | | | of radiation exposure for | |
| | | | LDCT is lower than chest | |
| | | | x-ray) | |
| False Negative | Patient thinks their lung | [ ] Lower | Patient may be more | [ ] Low |
| Result | cancer risk is lower than | [✓] Equivalent | reluctant to adhere to | [✓] Med |
| (erroneous | actual risk | [ ] Higher | lung cancer screening | [ ] High |
| under- | | | guidelines which may | |
| prediction of | | | lead to delayed | |
| lung cancer | | | diagnosis. However, this | |
| risk) | | | test should not replace | |
| | | | compliance with annual | |
| | | | lung cancer screening | |
| Off Label Use | Physician uses this test | [ ] Lower | Delay in potentially | [✓] Low |
| | to screen for lung cancer | [✓] Equivalent | beneficial screening | [ ] Med |
| | | [ ] Higher | | [ ] High |
| | Physician uses this test | | Prediction results may be | [ ] Low |
| | in patients not in | | inaccurate | [✓] Med |
| | intended use population | | | [ ] High |
| | (e.g. non-smokers) | | | |

The presently disclosed test provides a novel and convenient method for health care providers to assess and monitor the risk for lung cancer.

The consequence of a false negative result from this test is moderate based on the risk of potential hazards but these consequences are comparable to the standard of care. A low or moderate risk from this test should not preclude or exclude standard of care treatment (e.g., recommended method and frequency of lung cancer screening per current guidelines) or be interpreted as a reason to stop or decrease standard of care treatment.

The consequence of a false positive result from this test is low. A false positive may lead to a health care provider to recommend a more invasive screening method (e.g., LDCT vs sputum cytology) or lifestyle changes directed at reducing known risk factors for lung cancer. While there are minor radiation exposure risks associated with LDCT screening (albeit several fold lower exposure than that of chest radiography methods) risk-benefit analysis studies have concluded the risks to be acceptable due to the substantial mortality reduction obtained with screening. This test need not be the sole source for decision making for screening.

In one aspect, model performance was compared against the ability of risk factors that go into lung cancer screening eligibility to accurately predict those at future risk for lung cancer. Since these risk factors which determine screening criteria are what are used in clinical practice, the NLST clinical model was chosen to reflect the best comparator. The NLST is the largest lung cancer screening clinical trial that has been completed to date and is the basis for USPSTF lung cancer screening eligibility guidelines. For the NLST trial individuals with age and smoking risk factors aligning with existing USPSTF guidelines (aged 55+, current or former smoker who had accumulated >=30 pack years and if a former smoker quit less than 15 years prior). (National Lung Screening Trial Research Team. “Reduced lung-cancer mortality with low-dose computed tomographic screening.” N Engl J Med 2011;365:395-409). NLST risk-factor based criteria model showed an AUC performance of 0.689 and 0.670 in two study groups. (Tammemagi, et al. “Selection criteria for lung-cancer screening.” N Engl J Med 2013;368:728-36). Therefore, an AUC≥0.689 was used as a performance threshold during model development for the lung cancer risk test.

Accordingly, the lung cancer risk tests disclosed herein met the minimum performance requirement as set forth in Table 3.

TABLE 3
Model Performance Requirements

| Validation Metric(s) | Performance Requirement |
|---|---|
| Area Under the Curve (AUC) | Model AUC at 5 years ≥0.689 |

In one embodiment, the number of biomarkers useful for a biomarker subset or panel is based on the sensitivity and specificity value for the particular combination of biomarker values. The terms “sensitivity” and “specificity” are used herein with respect to the ability to correctly classify an individual, based on one or more biomarker values detected in their biological sample, as having an increased risk of lung cancer within 5 years or not having increased relative risk of lung cancer within the same time period. “Sensitivity” indicates the performance of the biomarker(s) with respect to correctly classifying individuals that have increased risk of lung cancer. “Specificity” indicates the performance of the biomarker(s) with respect to correctly classifying individuals who do not have increased relative risk of lung cancer.

In an alternate method, scores may be reported on a continuous range, with a threshold of high, intermediate or low risk of lung cancer, with thresholds determined based on clinical findings.

In some embodiments, overall performance of a panel of one or more biomarkers is represented by the area-under-the-curve (AUC) value. The AUC value is derived from a receiver operating characteristic (ROC) curve. The ROC curve is the plot of the true positive rate (sensitivity) of a test against the false positive rate (1-specificity) of the test. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., normal individuals and individuals at risk for lung cancer). ROC curves are useful for plotting the performance of a particular feature (e.g., any of the biomarkers described herein and/or any item of additional biomedical information) in distinguishing between two populations. Typically, the feature data across the entire population are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve.

Another factor that can affect the number of biomarkers to be used in a subset or panel of biomarkers is the procedures used to obtain biological samples from individuals who are being assessed for risk of lung cancer. In a carefully controlled sample procurement environment, the number of biomarkers necessary to meet desired sensitivity and specificity and/or threshold values will be lower than in a situation where there can be more variation in sample collection, handling and storage.

### Exemplary Uses of Biomarkers

In various exemplary embodiments, methods are provided for estimating or determining lung cancer risk by detecting one or more biomarker values corresponding to one or more biomarkers that are present in the circulation of an individual, such as in serum or plasma, by any number of analytical methods, including any of the analytical methods described herein.

In addition to testing biomarker levels as a stand-alone diagnostic test, biomarker levels can also be tested in conjunction with determination of SNPs or other genetic lesions or variability that are indicative of increased risk of susceptibility of disease or condition. (See, e.g., Amos et al., Nature Genetics 40, 616-622 (2009)).

In addition to testing biomarker levels as a stand-alone diagnostic test, biomarker levels can also be used in conjunction with screening methods, including lung imaging techniques, and more specifically, radiologic screening. Biomarker levels can also be used in conjunction with relevant symptoms or genetic testing. Detection of any of the biomarkers described herein may be useful to evaluate and/or to guide appropriate clinical care of the individual, whether the individual has healthy lung function or unhealthy lung function. In addition to testing biomarker levels in conjunction with relevant symptoms or risk factors, information regarding the biomarkers can also be evaluated in conjunction with other types of data, particularly data that indicates an individual's current state of lung health (e.g., patient clinical history, symptoms, family history, history of smoking or alcohol use, risk factors such as the presence of a genetic marker(s), and/or status of other biomarkers, etc.). These various data can be assessed by automated methods, such as a computer program/software, which can be embodied in a computer or other apparatus/device.

In addition to testing biomarker levels in conjunction with radiologic screening in high risk individuals (e.g., assessing biomarker levels in conjunction with detection of abnormal lung features), information regarding the biomarkers can also be evaluated in conjunction with other types of data, particularly data that indicates an individual's lung health (e.g., patient clinical history, symptoms, family history of lung disease, risk factors such as whether or not the individual is a smoker, heavy alcohol user and/or status of other biomarkers, etc.). These various data can be assessed by automated methods, such as a computer program/software, which can be embodied in a computer or other apparatus/device.

Any of the described biomarkers may also be used in imaging tests. For example, an imaging agent can be coupled to any of the described biomarkers, which can be used to aid in determining the state of lung health and also the presence or absence of abnormal lung function, to monitor response to therapeutic interventions, to select for target populations in a clinical trial among other uses.

### Detection and Determination of Biomarkers and Biomarker Values

A biomarker value for the biomarkers described herein can be detected using any of a variety of known analytical methods. In one embodiment, a biomarker value is detected using a capture reagent. As used herein, a “capture agent” or “capture reagent” refers to a molecule that is capable of binding specifically to a biomarker. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to SOMAmers, antibodies, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, imprinted polymers, avimers, peptidomimetics, a hormone receptor, a cytokine receptor, and synthetic receptors, and modifications and fragments of these.

In some embodiments, a biomarker value is detected using a biomarker/capture reagent complex.

In other embodiments, the biomarker value is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, the biomarker value is detected directly from the

biomarker in a biological sample.

In one embodiment, the biomarkers are detected using a multiplexed format that allows for the simultaneous detection of two or more biomarkers in a biological sample. In one embodiment of the multiplexed format, capture reagents are immobilized, directly or indirectly, covalently or non-covalently, in discrete locations on a solid support. In another embodiment, a multiplexed format uses discrete solid supports where each solid support has a unique capture reagent associated with that solid support, such as, for example quantum dots. In another embodiment, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one of multiple biomarkers to be detected in a biological sample.

In one or more of the foregoing embodiments, a fluorescent tag can be used to label a component of the biomarker/capture complex to enable the detection of the biomarker value. In various embodiments, the fluorescent label can be conjugated to a capture reagent specific to any of the biomarkers described herein using known techniques, and the fluorescent label can then be used to detect the corresponding biomarker value. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In one embodiment, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In other embodiments, the dye molecule includes a first type and a second type of dye molecule, such as, e.g., two different AlexaFluor molecules. In other embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more of the foregoing embodiments, a chemiluminescence tag can optionally be used to label a component of the biomarker/capture complex to enable the detection of a biomarker value. Suitable chemiluminescent materials include any of oxalyl chloride, Rodamin 6G, Ru (bipy) 32+, TMAE (tetrakis (dimethylamino) ethylene), Pyrogallol (1,2,3-trihydroxibenzene), Lucigenin, peroxyoxalates, Aryl oxalates, Acridinium esters, dioxetanes, and others.

In yet other embodiments, the detection method includes an enzyme/substrate combination that generates a detectable signal that corresponds to the biomarker value. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In yet other embodiments, the detection method can be a combination of fluorescence, chemiluminescence, radionuclide or enzyme/substrate combinations that generate a measurable signal. Multimodal signaling could have unique and advantageous characteristics in biomarker assay formats.

More specifically, the biomarker values for the biomarkers described herein can be detected using known analytical methods including, singleplex SOMAmer assays, multiplexed SOMAmer assays, singleplex or multiplexed immunoassays, mRNA expression profiling, miRNA expression profiling, mass spectrometric analysis, histological/cytological methods, etc. as detailed below.

Determination of Biomarker Values using SOMAmer-Based Assays

Assays directed to the detection and quantification of physiologically significant molecules in biological samples and other samples are important tools in scientific research and in the health care field. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. Nos. 6,242,246, 6,458,543, and 6,503,715, each of which is entitled “Nucleic Acid Ligand Diagnostic Biochip”. Once the microarray is contacted with a sample, the aptamers bind to their respective target molecules present in the sample and thereby enable a determination of a biomarker value corresponding to a biomarker.

As used herein, an “aptamer” refers to a nucleic acid that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Any of the aptamer methods disclosed herein can include the use of two or more aptamers that specifically bind the same target molecule. As further described below, an aptamer may include a tag. If an aptamer includes a tag, all copies of the aptamer need not have the same tag. Moreover, if different aptamers each include a tag, these different aptamers can have either the same tag or a different tag.

An aptamer can be identified using any known method, including the SELEX process. Once identified, an aptamer can be prepared or synthesized in accordance with any known method, including chemical synthetic methods and enzymatic synthetic methods.

As used herein, a “SOMAmer” or Slow Off-Rate Modified Aptamer refers to an aptamer having improved off-rate characteristics. SOMAmers can be generated using the improved SELEX methods described in U.S. Publication No. 2009/0004667, entitled “Method for Generating Aptamers with Improved Off-Rates.”

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication 20090098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Patent Application Publication 20090004667, entitled “Method for Generating Aptamers with Improved Off-Rates”, which describes improved SELEX methods for generating aptamers that can bind to target molecules. As mentioned above, these slow off-rate aptamers are known as “SOMAmers.” Methods for producing aptamers or SOMAmers and photoaptamers or SOMAmers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers or SOMAmers with improved off-rate performance.

A variation of this assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker value corresponding to a biomarker in the test sample.

In both of these assay formats, the aptamers or SOMAmers are immobilized on the solid support prior to being contacted with the sample. Under certain circumstances, however, immobilization of the aptamers or SOMAmers prior to contact with the sample may not provide an optimal assay. For example, pre-immobilization of the aptamers or SOMAmers may result in inefficient mixing of the aptamers or SOMAmers with the target molecules on the surface of the solid support, perhaps leading to lengthy reaction times and, therefore, extended incubation periods to permit efficient binding of the aptamers or SOMAmers to their target molecules. Further, when photoaptamers or photoSOMAmers are employed in the assay and depending upon the material utilized as a solid support, the solid support may tend to scatter or absorb the light used to effect the formation of covalent bonds between the photoaptamers or photoSOMAmers and their target molecules. Moreover, depending upon the method employed, detection of target molecules bound to their aptamers or photoSOMAmers can be subject to imprecision, since the surface of the solid support may also be exposed to and affected by any labeling agents that are used. Finally, immobilization of the aptamers or SOMAmers on the solid support generally involves an aptamer or SOMAmer-preparation step (i.e., the immobilization) prior to exposure of the aptamers or SOMAmers to the sample, and this preparation step may affect the activity or functionality of the aptamers or SOMAmers.

SOMAmer assays that permit a SOMAmer to capture its target in solution and then employ separation steps that are designed to remove specific components of the SOMAmer-target mixture prior to detection have also been described (see U.S. Patent Application Publication 20090042206, entitled “Multiplexed Analyses of Test Samples”). The described SOMAmer assay methods enable the detection and quantification of a non-nucleic acid target (e.g., a protein target) in a test sample by detecting and quantifying a nucleic acid (i.e., a SOMAmer). The described methods create a nucleic acid surrogate (i.e, the SOMAmer) for detecting and quantifying a non-nucleic acid target, thus allowing the wide variety of nucleic acid technologies, including amplification, to be applied to a broader range of desired targets, including protein targets.

SOMAmers can be constructed to facilitate the separation of the assay components from a SOMAmer biomarker complex (or photoSOMAmer biomarker covalent complex) and permit isolation of the SOMAmer for detection and/or quantification. In one embodiment, these constructs can include a cleavable or releasable element within the SOMAmer sequence. In other embodiments, additional functionality can be introduced into the SOMAmer, for example, a labeled or detectable component, a spacer component, or a specific binding tag or immobilization element. For example, the SOMAmer can include a tag connected to the SOMAmer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to a SOMAmer, thereby allowing for the release of the SOMAmer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. For estimation or determination of the risk of lung cancer, the molecular capture reagents can be an aptamer (e.g., modified aptamer or SOMAmer reagent) or an antibody or the like and the specific target would be a biomarker as in Table 6.

In one embodiment, a method for signal generation takes advantage of anisotropy signal change due to the interaction of a fluorophore-labeled capture reagent with its specific biomarker target. When the labeled capture reagent reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based SOMAmer assay that can be used to detect a biomarker value corresponding to a biomarker in a biological sample includes the following: (a) preparing a mixture by contacting the biological sample with a SOMAmer that includes a first tag and has a specific affinity for the biomarker, wherein a SOMAmer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the SOMAmer affinity complex; (e) releasing the SOMAmer affinity complex from the first solid support; (f) exposing the released SOMAmer affinity complex to a second solid support that includes a second capture element and allowing the second tag to associate with the second capture element; (g) removing any non-complexed SOMAmer from the mixture by partitioning the non-complexed SOMAmer from the SOMAmer affinity complex; (h) eluting the SOMAmer from the solid support; and (i) detecting the biomarker by detecting the SOMAmer component of the SOMAmer affinity complex.

Any means known in the art can be used to detect a biomarker value by detecting the SOMAmer component of a SOMAmer affinity complex. A number of different detection methods can be used to detect the SOMAmer component of an affinity complex, such as, for example, hybridization assays, mass spectroscopy, or QPCR. In some embodiments, nucleic acid sequencing methods can be used to detect the SOMAmer component of a SOMAmer affinity complex and thereby detect a biomarker value. Briefly, a test sample can be subjected to any kind of nucleic acid sequencing method to identify and quantify the sequence or sequences of one or more SOMAmers present in the test sample. In some embodiments, the sequence includes the entire SOMAmer molecule or any portion of the molecule that may be used to uniquely identify the molecule. In other embodiments, the identifying sequencing is a specific sequence added to the SOMAmer; such sequences are often referred to as “tags,” “barcodes,” or “zipcodes.” In some embodiments, the sequencing method includes enzymatic steps to amplify the SOMAmer sequence or to convert any kind of nucleic acid, including RNA and DNA that contain chemical modifications to any position, to any other kind of nucleic acid appropriate for sequencing.

In some embodiments, the sequencing method includes one or more cloning steps. In other embodiments the sequencing method includes a direct sequencing method without cloning.

In some embodiments, the sequencing method includes a directed approach with specific primers that target one or more SOMAmers in the test sample. In other embodiments, the sequencing method includes a shotgun approach that targets all SOMAmers in the test sample.

In some embodiments, the sequencing method includes enzymatic steps to amplify the molecule targeted for sequencing. In other embodiments, the sequencing method directly sequences single molecules. An exemplary nucleic acid sequencing-based method that can be used to detect a biomarker value corresponding to a biomarker in a biological sample includes the following: (a) converting a mixture of SOMAmers that contain chemically modified nucleotides to unmodified nucleic acids with an enzymatic step; (b) shotgun sequencing the resulting unmodified nucleic acids with a massively parallel sequencing platform such as, for example, the 454 Sequencing System (454 Life Sciences/Roche), the Illumina Sequencing System (Illumina), the ABI SOLID Sequencing System (Applied Biosystems), the HeliScope Single Molecule Sequencer (Helicos Biosciences), or the Pacific Biosciences Real Time Single-Molecule Sequencing System (Pacific BioSciences) or the Polonator G Sequencing System (Dover Systems); and (c) identifying and quantifying the SOMAmers present in the mixture by specific sequence and sequence count.

Determination of Biomarker Values using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I125) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

Determination of Biomarker Values using Gene Expression Profiling

Measuring mRNA in a biological sample may be used as a surrogate for detection of the level of the corresponding protein in the biological sample. Thus, any of the biomarkers or biomarker panels described herein can also be detected by detecting the appropriate RNA.

mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

miRNA molecules are small RNAs that are non-coding but may regulate gene expression. Any of the methods suited to the measurement of mRNA expression levels can also be used for the corresponding miRNA. Recently many laboratories have investigated the use of miRNAs as biomarkers for disease. Many diseases involve wide-spread transcriptional regulation, and it is not surprising that miRNAs might find a role as biomarkers. The connection between miRNA concentrations and disease is often even less clear than the connections between protein levels and disease, yet the value of miRNA biomarkers might be substantial. Of course, as with any RNA expressed differentially during disease, the problems facing the development of an in vitro diagnostic product will include the requirement that the miRNAs survive in the diseased cell and are easily extracted for analysis, or that the miRNAs are released into blood or other matrices where they must survive long enough to be measured. Protein biomarkers have similar requirements, although many potential protein biomarkers are secreted intentionally at the site of pathology and function, during disease, in a paracrine fashion. Many potential protein biomarkers are designed to function outside the cells within which those proteins are synthesized.

### Detection of Biomarkers Using In Vivo Molecular Imaging Technologies

Any of the described biomarkers (see Table 6) may also be used in molecular imaging tests. For example, an imaging agent can be coupled to any of the described biomarkers, which can be used to aid in estimation of determination of the risk of lung cancer, to monitor response to therapeutic interventions, to select a population for clinical trials among other uses.

In vivo imaging technologies provide non-invasive methods for determining the state of a particular disease or condition in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the lung cancer risk of an individual.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as a SOMAmer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning (coronary calcium score), positron emission tomography (PET), single photon emission computed tomography (SPECT), computed tomography angiography, and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18 Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Labeled antibodies which specifically bind any of the biomarkers in Table 6 can be injected into an individual being assessed for lung cancer risk, detectable according to the particular biomarker used, for the purpose of diagnosing or evaluating the disease risk of the individual. The label used will be selected in accordance with the imaging modality to be used, as previously described. Localization of the label permits determination of the tissue damage or other indications related to lung cancer. The amount of label within an organ or tissue also allows determination of the involvement of the lung cancer biomarkers in that organ or tissue.

Similarly, SOMAmers may be used for such in vivo imaging diagnostic methods. For example, a SOMAmer that was used to identify a particular biomarker described in Table 6 (and therefore binds specifically to that particular biomarker) may be appropriately labeled and injected into an individual being evaluated for lung cancer risk, detectable according to the particular biomarker, for the purpose of diagnosing or evaluating the levels of tissue damage, components of inflammatory response and other factors associated with the lung cancer risk in the individual. The label used will be selected in accordance with the imaging modality to be used, as previously described. Localization of the label permits determination of the site of the processes leading to increased risk. The amount of label within an organ or tissue also allows determination of the infiltration of the pathological process in that organ or tissue. SOMAmer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

The use of in vivo molecular biomarker imaging is increasing, including for clinical trials, for example, to more rapidly measure clinical efficacy in trials for new disease or condition therapies and/or to avoid prolonged treatment with a placebo for those diseases, such as multiple sclerosis, in which such prolonged treatment may be considered to be ethically questionable.

For a review of other techniques, see N. Blow, Nature Methods, 6, 465-469, 2009.

Determination of Biomarker Values using Mass Spectrometry Methods

A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS) N, atmospheric pressure photoionization mass spectrometry (APPI-MS), APPI-MS/MS, and APPI-(MS) N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to SOMAmers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

Determination of Biomarker Values using a Proximity Ligation Assay

A proximity ligation assay can be used to determine biomarker values. Briefly, a test sample is contacted with a pair of affinity probes that may be a pair of antibodies or a pair of SOMAmers, with each member of the pair extended with an oligonucleotide. The targets for the pair of affinity probes may be two distinct determinates on one protein or one determinate on each of two different proteins, which may exist as homo-or hetero-multimeric complexes. When probes bind to the target determinates, the free ends of the oligonucleotide extensions are brought into sufficiently close proximity to hybridize together. The hybridization of the oligonucleotide extensions is facilitated by a common connector oligonucleotide which serves to bridge together the oligonucleotide extensions when they are positioned in sufficient proximity. Once the oligonucleotide extensions of the probes are hybridized, the ends of the extensions are joined together by enzymatic DNA ligation.

Each oligonucleotide extension comprises a primer site for PCR amplification. Once the oligonucleotide extensions are ligated together, the oligonucleotides form a continuous DNA sequence which, through PCR amplification, reveals information regarding the identity and amount of the target protein, as well as, information regarding protein-protein interactions where the target determinates are on two different proteins. Proximity ligation can provide a highly sensitive and specific assay for real-time protein concentration and interaction information through use of real-time PCR. Probes that do not bind the determinates of interest do not have the corresponding oligonucleotide extensions brought into proximity and no ligation or PCR amplification can proceed, resulting in no signal being produced.

The foregoing assays enable the detection of biomarker values that are useful in methods for determining or estimating lung cancer risk, where the methods comprise detecting, in a biological sample from an individual, biomarker values that each correspond to a biomarker selected from the group consisting of the biomarkers provided in Table 6, wherein an assessment, as described in detail below, using the biomarker values indicates the risk of lung cancer in the individual. While certain of the described lung cancer risk biomarkers are useful alone for estimating or determining lung cancer risk, methods are also described herein for the grouping of multiple subsets of the lung cancer risk biomarkers that are each useful as a panel of three or more biomarkers. In accordance with any of the methods described herein, biomarker values can be detected and evaluated individually or they can be detected and evaluated collectively, as for example in a multiplex assay format.

A biomarker “signature” for a given diagnostic or predictive test contains a set of markers, each marker having different levels in the populations of interest. Different levels, in this context, may refer to different means of the marker levels for the individuals in two or more groups, or different variances in the two or more groups, or a combination of both. For the simplest form of a diagnostic test, markers can be used to assign an unknown sample from an individual into one of two groups, either lung cancer risk or not. The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker values. In general, classification methods are most easily performed using supervised learning techniques where a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging, boosting, forests and random forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009; each of which is incorporated by reference in its entirety.

To produce a classifier using supervised learning techniques, a set of samples called training data are obtained. In the context of diagnostic tests, training data includes samples from the distinct groups (classes) to which unknown samples will later be assigned. For example, samples collected from individuals in a control population and individuals in a particular disease, condition or event population can constitute training data to develop a classifier that can classify unknown samples (or, more particularly, the individuals from whom the samples were obtained) as either having the disease, condition or elevated risk of an event or being free from the disease, condition or elevated risk of an event. The development of the classifier from the training data is known as training the classifier. Specific details on classifier training depend on the nature of the supervised learning technique (see, e.g., Pattern Classification, R. O. Duda, et al., editors, John Wiley & Sons, 2nd edition, 2001; see also, The Elements of Statistical Learning-Data Mining, Inference, and Prediction, T. Hastie, et al., editors, Springer Science+Business Media, LLC, 2nd edition, 2009).

Since typically there are many more potential biomarker values than samples in a training set, care must be used to avoid over-fitting. Over-fitting occurs when a statistical model describes random error or noise instead of the underlying relationship. Over-fitting can be avoided in a variety of ways, including, for example, by limiting the number of markers used in developing the classifier, by assuming that the marker responses are independent of one another, by limiting the complexity of the underlying statistical model employed, and by ensuring that the underlying statistical model conforms to the data.

In order to identify a set of biomarkers associated with occurrence of events, the combined set of control and early event samples were analyzed using Principal Component Analysis (PCA). PCA displays the samples with respect to the axes defined by the strongest variations between all the samples, without regard to the case or control outcome, thus mitigating the risk of overfitting the distinction between case and control. Since the occurrence of lung cancer has a strong component of chance involved one would not expect to see a clear separation between the control and event sample sets. While the observed separation between case and control is not large, it occurs on the second principal component, corresponding to around 10% of the total variation in this set of samples, which indicates that the underlying biological variation is relatively simple to quantify.

In the next set of analyses, biomarkers can be analyzed for those components of difference between samples which were specific to the separation between the control samples and early event samples. One method that may be employed is the use of DSGA (Bair, E. and Tibshirani, R. (2004) Semi-supervised methods to predict patient survival from gene expression data. PLOS Biol., 2, 511-522) to remove (deflate) the first three principal component directions of variation between the samples in the control set. Although the dimensionality reduction is performed on the control set to discover, both the samples in the control and the samples from the early event samples are run through the PCA. Separation of cases from early events can be observed along the horizontal axis.

### Cross Validated Selection of Proteins Relevant to Lung Cancer Risk Estimation or Determination

In order to avoid over-fitting of protein predictive power to idiosyncratic features of a particular selection of samples, a cross-validation and dimensional reduction approach can be taken. Cross-validation involves the multiple selection of sets of samples to determine the association of risk by protein combined with the use of the unselected samples to monitor the ability of the method to apply to samples which were not used in producing the model of risk (The Elements of Statistical Learning-Data Mining, Inference, and Prediction, T. Hastie, et al., editors, Springer Science+Business Media, LLC, 2nd edition, 2009). We applied the supervised PCA method of Tibshirani et al (Bair, E. and Tibshirani, R. (2004) Semi-supervised methods to predict patient survival from gene expression data. PLOS Biol., 2, 511-522.) which is applicable to high dimensional datasets in the modeling of risk of lung cancer. The supervised PCA (SPCA) method involves the univariate selection of a set of proteins statistically associated with the observed event hazard in the data and the determination of the correlated component which combines information from all of these proteins. This determination of the correlated component is a dimensionality reduction step which not only combines information across proteins, but also mitigates the likelihood of overfitting by reducing the number of independent variables from the full protein menu of over 1000 proteins down to a few principal components (in this work, we only examined the first principal component).

### Univariate Analysis and Multivariate Analysis of the Relationship of Individual Proteins to Time to Event

The Cox proportional hazard model (Cox, David R (1972). “Regression Models and Life-Tables”. Journal of the Royal Statistical Society. Series B (Methodological) 34 (2): 187-220.)) is widely used in medical statistics. Cox regression avoids fitting a specific function of time to the cumulative survival, and instead employs a model of relative risk referred to a baseline hazard function (which may vary with time). The baseline hazard function describes the common shape of the survival time distribution for all individuals, while the relative risk gives the level of the hazard for a set of covariate values (such as a single individual or group), as a multiple of the baseline hazard. The relative risk is constant with time in the Cox model.

Accelerated failure time (AFT) models are a sub-class of survival models. Survival models predict time-to-event data under partial information. For example, in the data for the lung cancer model, the event is lung cancer diagnosis, but time-to-diagnosis event data is available for a fraction of the subjects in the study. For the rest of the subjects, the available information is that the subjects were not diagnosed with lung cancer from the time of the blood draw up to the end of the study. This second category is partial information, called “censoring”, because it is uncertain if or when they would ever be diagnosed with lung cancer.

Because survival models account for censoring, they can still use the data from those censored subjects, where other longitudinal models trying to predict when an event occurs can only use the information from subjects with lung cancer diagnoses. And because survival models take into account time-to-event, they can produce predicted probabilities of the event occurring within any time frame, which is different from most classification models (logistic regression, random forest).

AFT survival models in particular are a regression model which specifies/assumes a linear relationship between the model's covariates and log (time-to-event). So, a subject with 2× higher covariates (protein RFU counts) than baseline may be predicted to “survive” a lung cancer diagnosis 2× longer than baseline.

The two most common survival models are AFT models and proportional hazards models, and an AFT Weibull model is both. The definition of a proportional hazards model is a little more complicated than that of an AFT model-in a proportional hazards model, a subject with 2× higher covariates than baseline may have a 2× higher hazard at any time point, where hazard is the negative derivative of the survival curve over time.

Other common proportional hazards models are exponential and Cox models. Exponential models are a sub-type of Weibull model. Cox models are more limited in use-predicted probabilities of time-to-event are not available from Cox models, only relative risk. AFT models can give both absolute and relative risk.

### Kits

Any combination of the biomarkers of Table 6 can be detected using a suitable kit, such as for use in performing the methods disclosed herein. Furthermore, any kit can contain one or more detectable labels as described herein, such as a fluorescent moiety, etc.

In one embodiment, a kit includes (a) one or more capture reagents (such as, for example, at least one SOMAmer or antibody) for detecting one or more biomarkers in a biological sample, wherein the biomarkers include any of the biomarkers set forth in Table 6 and optionally (b) one or more software or computer program products for computing risk of lung cancer. Alternatively, rather than one or more computer program products, one or more instructions for manually performing the above steps by a human can be provided.

The combination of a solid support with a corresponding capture reagent having a signal generating material is referred to herein as a “detection device” or “kit”. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, antibody capture agents, positive control samples, negative control samples, software and information such as protocols, guidance and reference data.

In one aspect, the invention provides kits for the analysis of lung cancer risk. The kits include PCR primers for one or more SOMAmers specific to biomarkers selected from Table 6. The kit may further include instructions for use and correlation of the biomarkers with an estimation or determination of lung cancer risk. The kit may also include a DNA array containing the complement of one or more of the aptamers or SOMAmer reagents specific for the biomarkers selected from Table 6, reagents, and/or enzymes for amplifying or isolating sample DNA. The kits may include reagents for real-time PCR, for example, TaqMan probes and/or primers, and enzymes.

For example, a kit can comprise (a) reagents comprising at least capture reagent for quantifying one or more biomarkers in a test sample, wherein said biomarkers comprise the biomarkers set forth in Table 6, or any other biomarkers or biomarkers panels described herein, and optionally (b) one or more algorithms or computer programs for performing the steps of comparing the amount of each biomarker quantified in the test sample to one or more predetermined cutoffs and assigning a score for each biomarker quantified based on said comparison, combining the assigned scores for each biomarker quantified to obtain a total score, comparing the total score with a predetermined score, and using said comparison to determine whether an individual is at risk of lung cancer. Alternatively, rather than one or more algorithms or computer programs, one or more instructions for manually performing the above steps by a human can be provided.

### Computer Methods and Software

Once a biomarker or biomarker panel is selected, a method for diagnosing an individual can comprise the following: 1) collect or otherwise obtain a biological sample; 2) perform an analytical method to detect and measure the biomarker or biomarkers in the panel in the biological sample; 3) perform any data normalization or standardization required for the method used to collect biomarker values; 4) calculate the marker score; 5) combine the marker scores to obtain a total diagnostic or predictive score; and 6) report the individual's diagnostic or predictive score. In this approach, the diagnostic or predictive score may be a single number determined from the sum of all the marker calculations that is compared to a preset threshold value that is an indication of the presence or absence of disease. Or the diagnostic or predictive score may be a series of bars that each represent a biomarker value and the pattern of the responses may be compared to a pre-set pattern for determination of the presence or absence of disease, condition or the increased risk (or not) of an event.

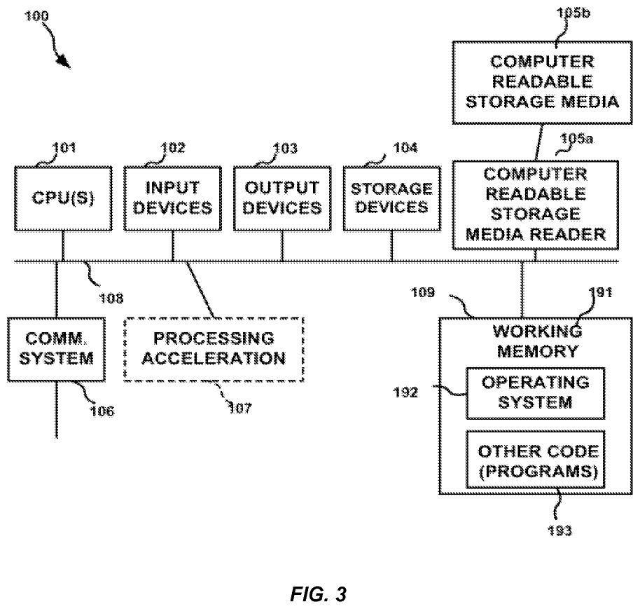

At least some embodiments of the methods described herein can be implemented with the use of a computer. An example of a computer system 100 is shown in FIG. 3. With reference to FIG. 3, system 100 is shown comprised of hardware elements that are electrically coupled via bus 108, including a processor 101, input device 102, output device 103, storage device 104, computer-readable storage media reader 105a, communications system 106, processing acceleration (e.g., DSP or special-purpose processors) 107 and memory 109. Computer-readable storage media reader 105a is further coupled to computer-readable storage media 105b, the combination comprehensively representing remote, local, fixed and/or removable storage devices plus storage media, memory, etc. for temporarily and/or more permanently containing computer-readable information, which can include storage device 104, memory 109 and/or any other such accessible system 100 resource. System 100 also comprises software elements (shown as being currently located within working memory 191) including an operating system 192 and other code 193, such as programs, data and the like.

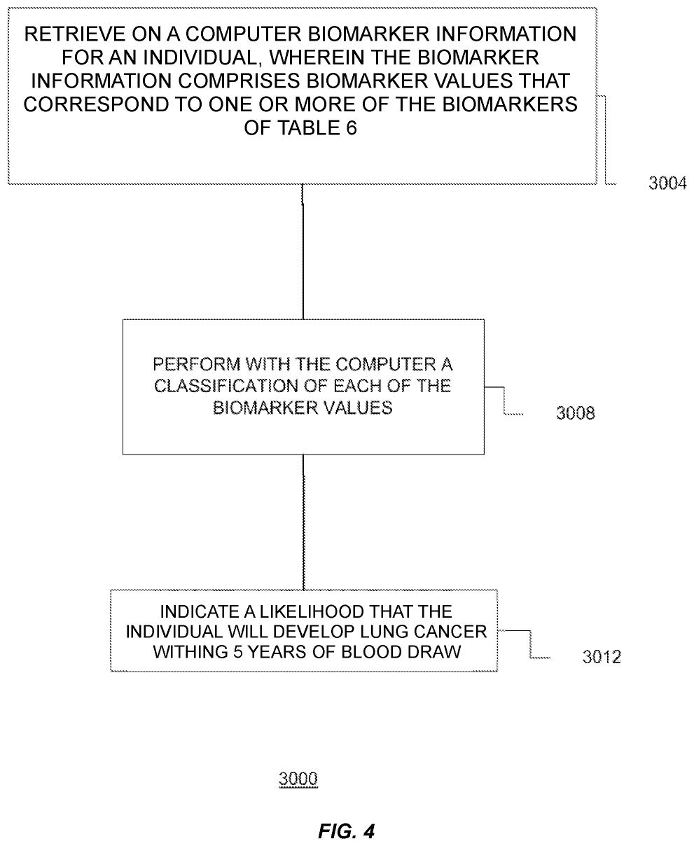

With respect to FIG. 4, system 100 has extensive flexibility and configurability. Thus, for example, a single architecture might be utilized to implement one or more servers that can be further configured in accordance with currently desirable protocols, protocol variations, extensions, etc. However, it will be apparent to those skilled in the art that embodiments may well be utilized in accordance with more specific application requirements. For example, one or more system elements might be implemented as sub-elements within a system 100 component (e.g., within communications system 106). Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system can comprise a database containing features of biomarkers characteristic of estimating or determining risk of lung cancer. The biomarker data (or biomarker information) can be utilized as an input to the computer for use as part of a computer implemented method. The biomarker data can include the data as described herein.

In one aspect, the system further comprises one or more devices for providing input data to the one or more processors.

The system further comprises a memory for storing a data set of ranked data elements.

In another aspect, the device for providing input data comprises a detector for detecting the characteristic of the data element, e.g., such as a mass spectrometer or gene chip reader.

The system additionally may comprise a database management system. User requests or queries can be formatted in an appropriate language understood by the database management system that processes the query to extract the relevant information from the database of training sets.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include an operating system (e.g., UNIX or Linux) for executing instructions from a database management system. In one aspect, the operating system can operate on a global communications network, such as the internet, and utilize a global communications network server to connect to such a network.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language.

The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing lung cancer risk with biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The lung cancer risk assessment with the biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to lung cancer risk biomarkers. The computer program may comprise multiple modules performing various functions or operations, such as a processing module for processing raw data and generating supplemental data and an analysis module for analyzing raw data and supplemental data to generate assessment of lung cancer risk. Calculation of lung cancer risk may optionally comprise generating or collecting any other information, including additional biomedical information, regarding the condition of the individual relative to the disease, condition or event, identifying whether further tests may be desirable, or otherwise evaluating the health status of the individual.

Referring now to FIG. 4, an example of a method of utilizing a computer in accordance with principles of a disclosed embodiment can be seen. In FIG. 4, a flowchart 3000 is shown. In block 3004, biomarker information can be retrieved for an individual. The biomarker information can be retrieved from a computer database, for example, after testing of the individual's biological sample is performed. The biomarker information can comprise biomarker values that each correspond to one or more of the biomarkers of Table 6. In block 3008, a computer can be utilized to perform a computation with each of the biomarker values. And, in block 3012, an estimation or determination can be made regarding risk of lung cancer. The indication can be output to a display or other indicating device so that it is viewable by a person. Thus, for example, it can be displayed on a display screen of a computer or other output device.

Some embodiments described herein can be implemented so as to include a computer program product. A computer program product may include a computer readable medium having computer readable program code embodied in the medium for causing an application program to execute on a computer with a database.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one aspect, a computer program product is provided for the estimation of lung cancer risk. The computer program product includes a computer readable medium embodying program code executable by a processor of a computing device or system, the program code comprising: code that retrieves data attributed to a biological sample from an individual, wherein the data comprises biomarker values that each correspond to one or more of the biomarkers of Table 6; and code that executes a computational method that indicates lung cancer risk of the individual as a function of the biomarker values.

In still another aspect, a computer program product is provided for risk of lung cancer. The computer program product includes a computer readable medium embodying program code executable by a processor of a computing device or system, the program code comprising: code that retrieves data attributed to a biological sample from an individual, wherein the data comprises a biomarker value corresponding to one or more of the biomarkers of Table 6; and code that executes a computational method that indicates the risk of lung cancer as a function of the biomarker value.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, PLAs, or ASICs.

It is also envisioned that embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

It is also noted that many of the structures, materials, and acts recited herein can be recited as means for performing a function or step for performing a function. Therefore, it should be understood that such language is entitled to cover all such structures, materials, or acts disclosed within this specification and their equivalents, including the matter incorporated by reference.

The biomarker identification process, the utilization of the biomarkers disclosed herein, and the various methods for determining biomarker values are described in detail above with respect to evaluating risk of lung cancer. However, the application of the process, the use of identified biomarkers, and the methods for determining biomarker values are fully applicable to other specific types of diseases or medical conditions, or to the identification of individuals who may or may not be benefited by an ancillary medical treatment.

### Other Methods

In some embodiments, the biomarkers and methods described herein are used to determine a medical insurance premium or coverage decision and/or a life insurance premium or coverage decision. In some embodiments, the results of the methods described herein are used to determine a medical insurance premium and/or a life insurance premium. In some such instances, an organization that provides medical insurance or life insurance requests or otherwise obtains information concerning a subject's tobacco use status and uses that information to determine an appropriate medical insurance or life insurance premium for the subject. In some embodiments, the test is requested by, and paid for by, the organization that provides medical insurance or life insurance. In some embodiments, the test is used by the potential acquirer of a practice or health system or company to predict future liabilities or costs should the acquisition go ahead.

In some embodiments, the biomarkers and methods described herein are used to predict and/or manage the utilization of medical resources. In some such embodiments, the methods are not carried out for the purpose of such prediction, but the information obtained from the method is used in such a prediction and/or management of the utilization of medical resources. For example, a testing facility or hospital may assemble information from the present methods for many subjects in order to predict and/or manage the utilization of medical resources at a particular facility or in a particular geographic area.

### EXAMPLES

The following examples are provided for illustrative purposes only and are not intended to limit the scope of the application as defined by the appended claims. All examples described herein were carried out using standard techniques, which are well known and routine to those of skill in the art. Routine molecular biology techniques described in the following examples can be carried out as described in standard laboratory manuals, such as Sambrook et al., Molecular Cloning: A Laboratory Manual, 3rd. ed., Cold Spring Harbor Laboratory Press, Cold Spring Harbor, N.Y., (2001).

### Example 1

### Model Specification

1.1 Model result description. In certain aspects, the endpoint for these analyses are lung cancer time-to-event outcomes, which have two components.
1) Time, in number of days from the blood draw to a lung cancer diagnosis (as a primary cancer) or to exiting/completing the study.
2) A binary variable denoting whether a lung diagnosis was observed during the study period or not.

1.2 Final model information. In one aspect, the final model is a 7-feature (see Table 6), Accelerated Failure Time (AFT) Weibull survival model. The model was trained on the entire study period, with performance maximized at 5 years.

This model provides two predictions:
1) Absolute risk: This output is the absolute probability of not having a lung cancer diagnosis (heretofore Pr(LC-free), a value between 0 and 1, within 5 years. When delivered as a result and assessed by business rules, the resulting predicted probability will be subtracted from 1, to provide an absolute 5-year probability for a lung cancer diagnosis.
2) Relative risk: This is a continuous variable that is calculated using the absolute risk probability generated by the model (described above), divided by the “baseline” absolute probability (defined below) in the training cohort. This approach allows for the model risk probability prediction to be interpreted such that higher values indicate a greater likelihood of a lung cancer diagnosis within the next five years.

The baseline risk probability score represents the “average” person in the training cohort based on the model algorithm. A “baseline” individual is defined as an individual with model feature values set to zero. All features in the model are centered on the overall mean, which means a value of 0 for any given feature is equal to the mean (i.e., the average). The baseline value is calculated by setting all the features to zero and then generating the absolute risk probability on those “zeroed” features. As such, a score less than 1 represents lower than average risk and a score greater than 1 represents higher risk than average risk. The rate of lung cancer diagnosis in the “ever smokers” ARIC dataset is consistent with U.S. population lung cancer diagnosis event rate age-matched for the intended use population.

The baseline risk score in the training data is 0.0095 or 0.95%.

The stratification of absolute risk scores is based on quartiles, with the first two quartiles collapsed into one single risk bin. (Preliminary analyses on the training data did not show strong separation between the first 2 quartiles). The groupings therefore represent Q1+Q2, Q3, and Q4, as shown in the table below (Table 4), note that the baseline risk (0.0095) is close to the upper limit of Q2.

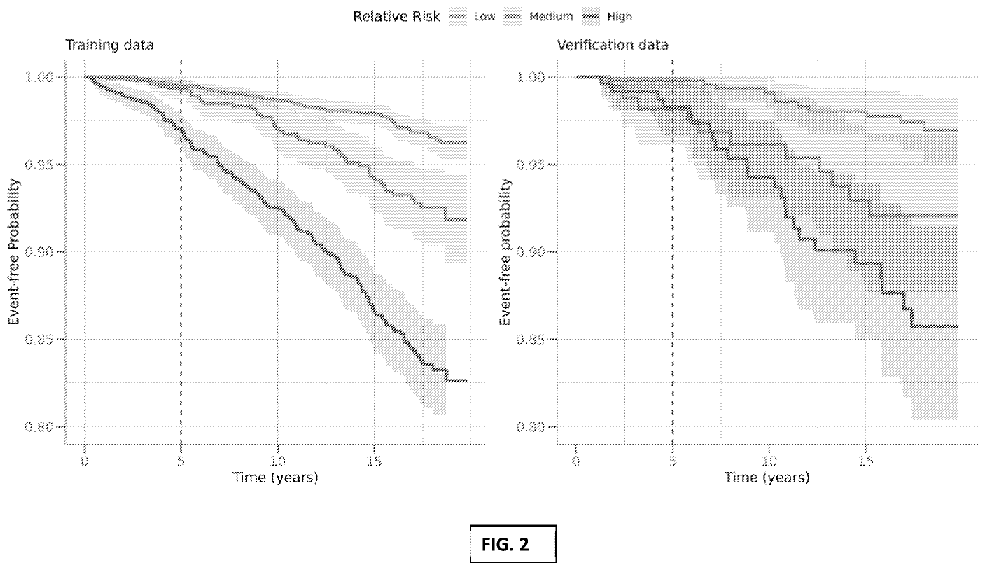

To support the reporting of lung cancer risk as a relative risk for LDT purposes, Kaplan-Meier plots, with subjects stratified into the three relative risk groups are shown in FIG. 2., with the summary of absolute probability and relative risk stratification and corresponding event rates shown in Table 4.

TABLE 4
Summary of observed event rate by absolute probability and relative risk bin in training data.

| | | | | | Total | | Number | 5-year |
|---|---|---|---|---|---|---|---|---|
| | | | | | Number | Lung | of Lung | Lung |
| | | | Bin Cutoffs | | of Lung | Cancer | Cancer | Cancer |
| Prediction | | Predicted | (in absolute | | Cancer | Event | events | event |
| Type | Bin | Class | probability) | N | Events | Rate | in 5 years | rate |
| Absolute | Q1 + | Low | 0 ≤ x ≤ | 2,129 | 55 | 2.6% | 8 | 0.38% |
| Risk | Q2 | | 0.00904 | | | | | |
| | Q3 | Medium | 0.00904 < x ≤ | 1,065 | 61 | 5.7% | 10 | 0.94% |
| | | | 0.01556 | | | | | |
| | Q4 | High | 0.01556 < x ≤ 1 | 1,065 | 133 | 12.5% | 32 | 3.00% |
| Relative | 0 ≤ x ≤ | Low | 0 ≤ x ≤ | 2,224 | 60 | 2.7% | 11 | 0.49% |
| Risk | 1 | | 0.00945 | | | | | |
| | 1 < x < | Medium | 0.00945 < x < | 795 | 44 | 5.5% | 5 | 0.63% |
| | 1.5 | | 0.0142 | | | | | |
| | x ≥ 1.5 | High | 0.0142 ≤ x ≤ 1 | 1,240 | 145 | 11.7% | 34 | 2.74% |

Based on this stratification, the scoring rules for relative risk for the lung cancer risk test are shown in Table 5.

TABLE 5
Scoring rules for relative risk

| Test Outcome × (relative risk | | |
|---|---|---|
| probability) | Predicted Class | Plain Language |
| 0 ≤ x ≤ 1 | Low | Relative risk predictions greater than or |
| | | equal to 0 and less than or equal to 1 are |
| | | labeled as “low” |
| 1 < x < 1.5 | Medium | Relative risk predictions greater than 1 and |
| | | less than 1.5 are labeled as “medium” |
| x ≥ 1.5 | High | Relative risk predictions greater than or |
| | | equal to 1.5 are labeled as “high” |

The git repository for model development can be found at “cancer-aric.”

TABLE 6
List of features in the final lung cancer risk model.

| Target Full Name | Target |
|---|---|
| Macrophage metalloelastase | MMP-12 |
| Pulmonary surfactant-associated | SP-D |
| protein D | |
| WAP four-disulfide core domain protein | HE4 |
| 2 | |
| Beta-microseminoprotein | PSP-94 |
| Pancreatic hormone | PH |
| Alpha-(1,3)-fucosyltransferase 5 | FUT5 |
| Cytokine receptor-like factor 1 | CRLF1 |

TABLE 7a
Lung Cancer Model Performance Data-Seven Analytes

| Full Model | | | | | | | Metric (e.g. AUC) |
|---|---|---|---|---|---|---|---|
| MMP-12 | SP-D | FUT5 | PSP-94 | HE4 | PH | CRLF1 | 0.76 |

TABLE 7b
Lung Cancer Model Performance Data-Single Analyte.

| Single Analyte Models | Metric (e.g. AUC) |
|---|---|
| PH | 0.68 |
| CRLF1 | 0.62 |
| PSP-94 | 0.67 |

TABLE 7c
Lung Cancer Model Performance Data-Two Analytes.

| Two Analyte Models | | Metric (e.g. AUC) |
|---|---|---|
| PH | CRLF1 | 0.68 |
| PH | PSP-94 | 0.69 |
| CRLF1 | PSP-94 | 0.67 |
| PH | MMP-12 | 0.7 |
| PH | SP-D | 0.72 |
| PH | FUT5 | 0.69 |
| PH | HE4 | 0.72 |
| CRLF1 | MMP-12 | 0.69 |
| CRLF1 | SP-D | 0.69 |
| CRLF1 | FUT5 | 0.66 |
| CRLF1 | HE4 | 0.7 |
| PSP-94 | MMP-12 | 0.71 |
| PSP-94 | SP-D | 0.72 |
| PSP-94 | FUT5 | 0.69 |
| PSP-94 | HE4 | 0.71 |

TABLE 7d
Lung Cancer Model Performance Data-Three Analytes.

| Three Analyte Models | | | Metric (e.g. AUC) |
|---|---|---|---|
| PH | CRLF1 | PSP-94 | 0.69 |
| PH | CRLF1 | MMP-12 | 0.71 |
| PH | CRLF1 | SP-D | 0.73 |
| PH | CRLF1 | FUT5 | 0.7 |
| PH | CRLF1 | HE4 | 0.72 |
| PH | PSP-94 | MMP-12 | 0.72 |
| PH | PSP-94 | SP-D | 0.74 |
| PH | PSP-94 | FUT5 | 0.71 |
| PH | PSP-94 | HE4 | 0.72 |
| CRLF1 | PSP-94 | MMP-12 | 0.71 |
| CRLF1 | PSP-94 | SP-D | 0.72 |
| CRLF1 | PSP-94 | FUT5 | 0.69 |
| CRLF1 | PSP-94 | HE4 | 0.71 |

TABLE 7e
Lung Cancer Model Performance Data-Four Analytes.

| Four Analyte Models | | | | Metric (e.g. AUC) |
|---|---|---|---|---|
| PH | CRLF1 | PSP-94 | MMP-12 | 0.73 |
| PH | CRLF1 | PSP-94 | SP-D | 0.74 |
| PH | CRLF1 | PSP-94 | FUT5 | 0.71 |
| PH | CRLF1 | PSP-94 | HE4 | 0.73 |

TABLE 7f
Lung Cancer Model Performance Data-Five Analytes.

| Five Analyte Models | | | | | Metric (e.g. AUC) |
|---|---|---|---|---|---|
| PH | CRLF1 | PSP-94 | MMP-12 | SP-D | 0.76 |
| PH | CRLF1 | PSP-94 | MMP-12 | FUT5 | 0.73 |
| PH | CRLF1 | PSP-94 | MMP-12 | HE4 | 0.74 |
| PH | CRLF1 | PSP-94 | SP-D | FUT5 | 0.74 |
| PH | CRLF1 | PSP-94 | SP-D | HE4 | 0.76 |
| PH | CRLF1 | PSP-94 | FUT5 | HE4 | 0.74 |
| PH | CRLF1 | PSP-94 | FUT5 | SP-D | 0.74 |

TABLE 7g
Lung Cancer Model Performance Data-Six Analytes.

| | | | | | | Metric |
|---|---|---|---|---|---|---|
| Six Analyte Models | | | | | | (e.g. AUC) |
| PH | CRLF1 | PSP-94 | MMP-12 | SP-D | FUT5 | 0.76 |
| PH | CRLF1 | PSP-94 | MMP-12 | SP-D | HE4 | 0.76 |
| PH | CRLF1 | PSP-94 | MMP-12 | FUT5 | HE4 | 0.74 |
| PH | CRLF1 | PSP-94 | SP-D | FUT5 | HE4 | 0.76 |
| PH | CRLF1 | PSP-94 | SP-D | FUT5 | MMP-12 | 0.76 |

1.3 In one aspect, the model output is the Pr(lung cancer-free) at five years. The output will be reported as the probability of a lung cancer diagnosis, which is (1-Pr(lung cancer-free). The event probability at five years will be reported as a continuous variable. Because the output of this model is a probability, values outside of the range [0,1] are failures and will not be reported.

Example: A hypothetical patient has an absolute predicted probability equal to 0.0100 based on the proteomic model. This patient's relative risk is 1.06, and putting them in the middle risk bin.

$$RR = \frac{0.01}{0.0095} = 1.6$$

This relative risk in the example is interpreted as follows: this patient has 1.06 times or a 6% higher risk for a lung cancer diagnosis within the next five years compared to the average individual in our reference population.

### Example 2

### Datasets for Test Development and Validation

2.1 Development and validation cohort(s). The Atherosclerosis Risk in Communities (ARIC) Study is a prospective epidemiologic study conducted in four U.S. communities: Forsyth County, NC; Jackson, MS; the northwest suburbs of Minneapolis, MN; and Washington County, MD. The ARIC study enrolled 15,792 participants aged 45-64. Enrollment took place from 1987 to 1989 and now has 30 years of follow-up through to study visit 6 in 2016-2017. While the ARIC Study was originally designed to investigate the etiology and natural history of atherosclerosis, the etiology of clinical atherosclerotic diseases, and variation in cardiovascular risk factors, medical care and disease by race, gender, location, and date, expansion of the study to facilitate cancer epidemiology research has been implemented. Cancer incidence and mortality were adjudicated through medical record, abstracted record, and cancer registry review, coupled with self-report information obtained during follow-up. (Joshu et al. “Enhancing the Infrastructure of the Atherosclerosis Risk in Communities (ARIC) Study for Cancer Epidemiology Research: ARIC Cancer.” Cancer Epidemiol Biomarkers Prev 2018;27:295-305). The lung cancer risk model has been developed from documented lung cancer diagnoses in ever smokers (defined as current or former smokers at visit 3) starting from the third visit conducted from 1993-1995 through a twenty-year follow-up period, however this model is intended to perform on lung cancer diagnoses up to 5 years of follow-up.

For the recent (2014-2018) U.S. population the 5-year risk rate of lung cancer in 40-74-year-old men and women regardless of smoking status is 0.715%. (“SEER*Explorer” National Cancer Institute. Surveillance, Epidemiology, and End Results Program. Available online at https://seer.cancer.gov/explorer). Which is consistent with the 0.759% 5-year incidence rate of lung cancer in the ARIC visit 3 dataset which spans men and women 50-73 years of age when accounting for both ever and never smokers, and an increased 5-year incidence rate of 1.23% in ever smokers.

2.2 Dataset Stratification. For this test, the data was split independently into training (70%), verification (15%), and validation (15%) sets stratified by lung cancer diagnosis as the endpoint, allowing identification of a robust model while mitigating overfitting issues. The validation data set was not used in the POC or refinement stages.

### 2.2.1 Model Training Data

TABLE 8
Demographic table for training data used for lung cancer model development
from “ever smokers” in ARIC visit 3 through to end of follow up period.

| | | | | | | Observed | |
|---|---|---|---|---|---|---|---|
| Covariate | Measure | Total | | Censored* | | Event | |
| Sample Size | N | 4260 | | 4011 | | 249 | |
| Gender | Female | 1811 | (42.5%) | 1724 | (43%) | 87 | (34.9%) |
| | Male | 2449 | (57.5%) | 2287 | (57%) | 162 | (65.1%) |
| Ethnicity | Black | 832 | (19.5%) | 792 | (19.7%) | 40 | (16.1%) |
| | White | 3428 | (80.5%) | 3219 | (80.3%) | 209 | (83.9%) |
| Tobacco Use | Current | 1268 | (29.8%) | 1133 | (28.2%) | 135 | (54.2%) |
| | Former | 2992 | (70.2%) | 2878 | (71.8%) | 114 | (45.8%) |
| Time to | Mean (SD) | 5129 | (2054) | 5225 | (2030) | 3591 | (1820) |
| Diagnosis/ | Median | 6285 | | 6334 | | 3614 | |
| Censoring | Range | 6-7230 | | 6-7230 | | 112-6843 | |
| Age | Mean (SD) | 60.06 | (5.69) | 59.95 | (5.675) | 61.9 | (5.632) |

| Median | 60 | 60 | 62 |
|---|---|---|---|
| Range | 50-73 | 50-73 | 50-71 |

*Observed event is lung cancer diagnosis. Censored individuals did not have an event (lung cancer diagnosis) through last follow-up.

TABLE 9
Cumulative lung cancer diagnosis event rate by 5-year time
intervals post visit 3 blood draw for training data.

| Status | Years 0-5 | Years 5-10 | Years 10-15 |
|---|---|---|---|
| Censored after time interval | 3792 | 3717 | 3059 |
| Censored during time interval* | 418 | 468 | 1077 |
| Event | 50 | 75 | 124 |
| Event rate | 1.17% | 1.76% | 2.91% |

*Individuals with last follow-up before end of time interval

### 2.2.2 Model Verification Data

TABLE 10
Demographic table for verification data used for lung cancer model development
from ever smokers in ARIC visit 3 through to end of follow up period.

| Covariate | Measure | Total | | Censored* | | Observed Event | |
|---|---|---|---|---|---|---|---|
| Sample Size | N | 913 | | 867 | | 46 | |
| Gender | Female | 401 | (43.9%) | 384 | (44.3%) | 17 | (37%) |
| | Male | 512 | (56.1%) | 483 | (55.7%) | 29 | (63%) |
| Ethnicity | Black | 181 | (19.8%) | 174 | (20.1%) | 7 | (15.2%) |
| | White | 732 | (80.2%) | 693 | (79.9%) | 39 | (84.8%) |
| Tobacco Use | Current | 280 | (30.7%) | 252 | (29.1%) | 28 | (60.9%) |
| | Former | 633 | (69.3%) | 615 | (70.9%) | 18 | (39.1%) |
| Time to Diagnosis/ | Mean (SD) | 5179 | (2018) | 5268 | (1996) | 3503 | (1691) |
| Censoring | Median | 6279 | | 6324 | | 3752 | |
| | Range | 10-7229 | | 10-7229 | | 473-6570 | |
| Age | Mean (SD) | 59.92 | (5.66) | 59.85 | (5.645) | 61.26 | (5.844) |

| Median | 60 | 60 | 61.5 |
|---|---|---|---|
| Range | 50-71 | 50-71 | 51-70 |

*Observed event is lung cancer diagnosis. Censored individuals did not have an event (lung cancer diagnosis) through last follow-up.

TABLE 11
Cumulative lung cancer diagnosis event rate by 5-year time
intervals post visit 3 blood draw for verification data.

| Status | Years 0-5 | Years 5-10 | Years 10-15 |
|---|---|---|---|
| Censored after time interval | 829 | 815 | 663 |
| Censored during time interval* | 76 | 84 | 226 |
| Event | 8 | 14 | 24 |
| Event rate | 0.88% | 1.53% | 2.63% |

*Individuals with last follow-up before end of time interval

### 2.2.3 Model Validation Data

TABLE 12
Demographic table for validation data to be used for lung cancer model validation
from ever smokers in ARIC visit 3 through to end of follow up period.

| Covariate | Measure | Total | | Censored* | | Observed Event | |
|---|---|---|---|---|---|---|---|
| Sample Size | N | 912 | | 859 | | 53 | |
| Gender | Female | 418 | (45.8%) | 393 | (45.8%) | 25 | (47.2%) |
| | Male | 494 | (54.2%) | 466 | (54.2%) | 28 | (52.8%) |
| Ethnicity | Black | 176 | (19.3%) | 165 | (19.2%) | 11 | (20.8%) |
| | White | 736 | (80.7%) | 694 | (80.8%) | 42 | (79.2%) |
| Tobacco Use | Current | 288 | (31.6%) | 255 | (29.7%) | 33 | (62.3%) |
| | Former | 624 | (68.4%) | 604 | (70.3%) | 20 | (37.7%) |
| Time to Diagnosis/ | Mean (SD) | 5185 | (2084) | 5325 | (2023) | 2914 | (1717) |

| Censoring | Median | 6324 | 6376 | 2731 |
|---|---|---|---|---|
| | Range | 30-7230 | 30-7230 | 244-6432 |

*Observed event is lung cancer diagnosis.
Censored individuals did not have an event (lung cancer diagnosis) through last follow-up.

TABLE 13
Cumulative lung cancer diagnosis event rate by 5-year time
intervals post visit 3 blood draw for validation data.

| Status | Years 0-5 | Years 5-10 | Years 10-15 |
|---|---|---|---|
| Censored after time interval | 813 | 795 | 663 |
| Censored during time interval* | 82 | 99 | 231 |
| Events | 17 | 18 | 18 |
| Event rate | 1.86% | 1.97% | 1.97% |

*Individuals with last follow-up before end of time interval

### Example 3

### Results from Development

### 3.1 Data QC and Pre-Analytics Results

The original clinical dataset included 11,288 samples. The following number of samples were removed based on various flags. The removals are detailed in Table 14.

TABLE 14
Samples removed from ARIC visit 3 for lung cancer
risk test development and validation.

| | N | N |
|---|---|---|
| Step | (removed) | (remaining) |
| Baseline | | 11,288 |
| Prevalent cancer | 969 | 10,319 |
| Never smokers | 4,181 | 6,122 |
| Outside range normalization scale factors | 15 | 6,107 |
| Outliers | 23 | 6,085* |
| Missing event/time | 0 | 6,085 |

Note:
Outlier are samples with >5% of SOMAmer measurements exceeding 6 median absolute deviations from the median.
RowCheck failures are samples with at least one scale factor outside the acceptable range of 0.4 to 2.5.
*There were 23 outliers, but one of them also fell outside the normalization scale factor range and had already been removed in the previous step.

Additionally, 363 analytes were removed before analyses began as they did not pass target confirmation specificity testing leaving 4921 analytes available for analyses. No other issues were identified during Data QC or Pre-Analytics.

The samples for this test were run on assay version 4.0 Master Mix Lot 1 from May 13, 2019 until Jul. 8, 2019.

3.2 Refinement Approach and Results. The final model for the Lung Cancer risk test contains 7 features and was developed using an AFT survival model using a Weibull distribution. The model was trained on 70% of the ARIC visit 3 dataset with no prevalent cancer and who have a history of either current or former tobacco smoking. Verification metrics were calculated on a separate 15% dataset, and an additional 15% dataset was held-out for use in validation.

Observations over the entire course of the study period were used, with model performance optimized for 5 years. The model was built using a reduced feature list of the top 100 univariate features, further refined by stability selection with upsampling of the event class. Features with high CV (CV>10%) were removed from the remaining analytes. A backwards feature selection process using AFT Weibull models was used to further refine features, to find the model with the best performance.

A refined model was further assessed using model hardening tools and was refined in order to ensure concordance between assay versions V4.0 and V4.1.

The final model was assessed for predicting a lung cancer diagnosis in ever smokers, using AUC at 5 years (1825 days). Additional metrics such as C-Index, PEC, sensitivity, and specificity were reported. The results for training and verification datasets are shown in Table 15.

TABLE 15
Performance metrics and 95% CI for the training and verification data from the
final lung cancer risk model in ever smokers, evaluated at 5 years (1825 days).

| | | AUC | Sensitivity | Specificity | | PEC |
|---|---|---|---|---|---|---|
| Dataset | N | at 5 years | at 5 years | at 5 years | C-Index* | at 5 years |
| Training | 4260 | 0.76 | 0.70 | 0.72 | 0.73 | 0.01 |
| | | (0.69-0.82) | (0.51-0.90) | (0.51-0.87) | (0.70-0.76) | (0.009-0.02) |
| Verification | 913 | 0.72 | 0.88 | 0.59 | 0.71 | 0.01 |
| | | (0.55-0.89) | (0.43-1) | (0.54-0.95) | (0.62-0.79) | (0.005-0.02) |

*C-Index is not time specific.

For informational purposes only, the final model was additionally used to predict the risk of a lung cancer diagnosis at 10-and 15-years post-blood draw in ever smokers, and performance metrics were calculated. The performance metrics are detailed in Table 16.

TABLE 16
Performance metrics for the lung cancer risk model in ever smokers,
evaluated at 10-and 15-years post-visit 3 blood draw.

| Dataset | Timepoint | AUC | Sensitivity | Specificity | C-Index | PEC |
|---|---|---|---|---|---|---|
| Training | 10 years | 0.752 | 0.672 | 0.714 | 0.732 | 0.035 |
| | 15 years | 0.771 | 0.793 | 0.617 | | 0.063 |
| Verification | 10 years | 0.729 | 0.818 | 0.588 | 0.707 | 0.029 |
| | 15 years | 0.726 | 0.816 | 0.596 | | 0.056 |

For investigational purposes only, the final model was additionally used to predict the risk of a lung cancer diagnosis in never smokers from ARIC visit 3 at 5-, 10- and 15-years post-blood draw, and performance metrics were calculated. The lung cancer event rates, and summary demographics in the never smoker dataset are detailed in Tables 17a and 17b, respectively. The performance metrics are detailed in Table 18.

TABLE 17a
Demographic table for never smokers in ARIC
visit 3 through to end of follow up period.

| | | | | | | Observed | |
|---|---|---|---|---|---|---|---|
| Covariate | Measure | Total | | Censored* | | Event | |
| Sample | N | 4151 | | 4126 | | 25 | |
| Size | | | | | | | |
| Gender | Female | 2875 | (69.3%) | 2857 | (69.2%) | 18 | (72%) |
| | Male | 1276 | (30.7%) | 1269 | (30.8%) | 7 | (28%) |
| Ethnicity | Black | 998 | (24%) | 989 | (24%) | 9 | (36%) |
| | White | 3153 | (76%) | 3137 | (76%) | 16 | (64%) |
| Time to | Mean | 5626 | (1857) | 5636 | (1853) | 3933 | (1654) |
| Diagnosis/ | (SD) | | | | | | |
| Censoring | Median | 6475 | | 6478 | | 3964 | |
| | Range | 11-7230 | | 11-7230 | | 742-6345 | |
| Age | Mean | 59.98 | (5.713) | 59.98 | (5.712) | 61.04 | (5.827) |
| | (SD) | | | | | | |

| | | | |
|---|---|---|---|
| Median | 60 | 60 | 61 |
| Range | 49-72 | 49-72 | 51-69 |

*Observed event is lung cancer diagnosis. Censored individuals did not have an event (lung cancer diagnosis) through last follow-up.

TABLE 17b
Cumulative lung cancer diagnosis event rate by 5-year time
intervals post visit 3 blood draw in never smokers.

| Status | Years 0-5 | Years 5-10 | Years 10-15 |
|---|---|---|---|
| Censored after time interval | 3856 | 3848 | 3426 |
| Censored during time interval* | 292 | 295 | 711 |
| Event | 3 | 8 | 14 |

*Individuals with last follow-up before end of time interval.

TABLE 18
Performance metrics for the lung cancer risk model in never smokers,
evaluated at 5-, 10- and 15-years post-visit 3 blood draw.

| Timepoint | AUC | Sensitivity | Specificity | C-Index | PEC |
|---|---|---|---|---|---|
| 5 years | 0.685 | 0.667 | 0.92 | 0.583 | 0.001 |
| | (0.5-0.98) | (0.5-1) | (0.84-0.98) | (0.41-0.68) | (0.0001-0.002) |
| 10 years | 0.621 | 0.455 | 0.867 | | 0.004 |
| 15 years | 0.557 | 0.381 | 0.872 | | 0.009 |

### Example 4

### Longitudinal Analysis of Lung Cancer Risk Prediction

Data from ARIC visits 2, 3, and 5 was used to analyze changes in lung cancer risk according to certain parameters over time. Specifically, changes in lung cancer risk with changes or consistencies in smoking status between ARIC Visits 2 and 3 (Aim 1), change in lung cancer risk over time between ARIC Visit 2 and 3 in subjects diagnosed with lung cancer differing proximities following Visit 3 (Aim 2), and the differences in lung cancer risk predictions between individuals with or without a prevalent lung cancer diagnosis at the time of blood draw at Visit 3 or Visit 5 (Aim 3) were assessed. ARIC Visit 2, 3, and 5 subject demographics are shown in Tables 19-22.

TABLE 19
Demographic table for ARIC Visit 2 participants without prevalent
cancer at the time of visit, stratified by incident lung cancer diagnosis
through to end of follow up period (752 individuals missing lung
cancer diagnosis information were excluded from this table).

| | | | | Lung Cancer Diagnosis | | | |
|---|---|---|---|---|---|---|---|
| Covariate | Measure | Total | | No | | Yes | |
| Sample Size | N | 10983 | | 10539 | | 444 | |
| Gender | Female | 6012 | (54.7%) | 5829 | (55.3%) | 183 | (41.2%) |
| | Male | 4971 | (45.3%) | 4710 | (44.7%) | 261 | (58.8%) |
| Ethnicity | African | 2628 | (23.9%) | 2532 | (24%) | 96 | (21.6%) |
| | American | | | | | | |
| | Caucasian | 8355 | (76.1%) | 8007 | (76%) | 348 | (78.4%) |
| Smoking | NA | 18 | (0.2%) | 16 | (0.2%) | 2 | (0.5%) |
| Status | Current | 2452 | (22.3%) | 2178 | (20.7%) | 274 | (61.7%) |
| | Former | 4175 | (38%) | 4034 | (38.3%) | 141 | (31.8%) |
| | Never | 4338 | (39.5%) | 4311 | (40.9%) | 27 | (6.1%) |
| Age | Mean (SD) | 57.022 | (5.729) | 56.963 | (5.724) | 58.423 | (5.685) |

| | | | |
|---|---|---|---|
| Median | 57 | 57 | 58 |
| Range | 46-70 | 46-70 | 47-68 |

TABLE 20
Demographic table for ARIC Visit 3 participants without prevalent
cancer at the time of visit, stratified by incident lung cancer diagnosis
through to end of follow up period (695 individuals missing lung
cancer diagnosis information were excluded from this table).

| | | | | Incident Lung Cancer Diagnosis | | | |
|---|---|---|---|---|---|---|---|
| Covariate | Measure | Total | | No | | Yes | |
| Sample Size | N | 10593 | | 10199 | | 394 | |
| Gender | Female | 5690 | (53.7%) | 5533 | (54.3%) | 157 | (39.8%) |
| | Male | 4903 | (46.3%) | 4666 | (45.7%) | 237 | (60.2%) |
| Ethnicity | African | 2265 | (21.4%) | 2195 | (21.5%) | 70 | (17.8%) |
| | American | | | | | | |
| | Caucasian | 8328 | (78.6%) | 8004 | (78.5%) | 324 | (82.2%) |
| Smoking | Current | 1895 | (17.9%) | 1692 | (16.6%) | 203 | (51.5%) |
| Status | Former | 4401 | (41.5%) | 4240 | (41.6%) | 161 | (40.9%) |
| | NA | 30 | (0.3%) | 28 | (0.3%) | 2 | (0.5%) |
| | Never | 4267 | (40.3%) | 4239 | (41.6%) | 28 | (7.1%) |
| Age | Mean (SD) | 60.086 | (5.695) | 60.019 | (5.689) | 61.799 | (5.595) |

| | | | |
|---|---|---|---|
| Median | 60 | 60 | 62 |
| Range | 49-73 | 49-73 | 50-71 |

TABLE 21
Demographic table for ARIC VTable 22isit 3 participants stratified
by prevalent lung cancer status (695 individuals missing lung
cancer diagnosis information were excluded from this table).

| | | | | Prevalent Lung Cancer | | | |
|---|---|---|---|---|---|---|---|
| Covariate | Measure | Total | | No | | Yes | |
| Sample Size | N | 10593 | | 10573 | | 20 | |
| Gender | Female | 5690 | (53.7%) | 5680 | (53.7%) | 10 | (50.0%) |
| | Male | 4903 | (46.3%) | 4893 | (46.3%) | 10 | (50.0%) |
| Ethnicity | African | 2265 | (21.4%) | 2263 | (21.4%) | 2 | (10.0%) |
| | American | | | | | | |
| | Caucasian | 8328 | (78.6%) | 8310 | (78.6%) | 18 | (90.0%) |
| Smoking | Current | 1895 | (17.9%) | 1888 | (17.9%) | 7 | (35.0%) |
| Status | Former | 4401 | (41.5%) | 4391 | (41.5%) | 10 | (50.0%) |
| | NA | 30 | (0.3%) | 30 | (0.3%) | 0 | (0.0%) |
| | Never | 4267 | (40.3%) | 4264 | (40.3%) | 3 | (15.0%) |
| Age | Mean | 60.086 | (5.695) | 60.077 | (5.693) | 64.85 | (4.793) |
| | (SD) | | | | | | |

| | | | |
|---|---|---|---|
| Median | 60 | 60 | 66.5 |
| Range | 49-73 | 49-73 | 53-71 |

TABLE 22
Demographic table for ARIC visit 5 participants stratified by
prevalent lung cancer status (300 individuals missing lung
cancer diagnosis information were excluded from this table).

| | | | | Prevalent Lung Cancer | | | |
|---|---|---|---|---|---|---|---|
| Covariate | Measure | Total | | No | | Yes | |
| Sample Size | N | 4954 | | 4910 | | 44 | |
| Gender | Female | 2778 | (56.1%) | 2756 | (56.1%) | 22 | (50%) |
| | Male | 2176 | (43.9%) | 2154 | (43.9%) | 22 | (50%) |
| Ethnicity | African | 952 | (19.2%) | 943 | (19.2%) | 9 | (20.5%) |
| | American | | | | | | |
| | Caucasian | 4002 | (80.8%) | 3967 | (80.8%) | 35 | (79.5%) |
| Smoking | Current | 268 | (5.4%) | 265 | (5.4%) | 3 | (6.8%) |
| Status | Former | 2315 | (46.7%) | 2286 | (46.6%) | 29 | (65.9%) |
| | NA | 553 | (11.2%) | 546 | (11.1%) | 7 | (15.9%) |
| | Never | 1818 | (36.7%) | 1813 | (36.9%) | 5 | (11.4%) |
| Age | Mean | 75.737 | (5.229) | 75.725 | (5.225) | 77.091 | (5.531) |
| | (SD) | | | | | | |

| | | | |
|---|---|---|---|
| Median | 75 | 75 | 77 |
| Range | 66-90 | 66-90 | 68-88 |

Data quality control (QC) pre-analytics was performed on ARIC visit 2, visit 3, and visit 5. There were 11,779 samples from visit 2, 11,360 samples from visit 3, and 5,281 samples from visit 5 with clinical and RFU data available for analysis.

Data QC showed that 28 (0.238%) samples from visit 2, 41 (0.361%) samples from visit 3, and 27 (0.511%) samples from visit 5 that were identified as outlier samples, defined as >5% of analytes exceed 6 median absolute deviations from the median. Data QC also showed that 17 (0.144%) samples from visit 2, 36 (0.317%) samples from visit 3, and 0 (0.0%) samples from visit 5 that failed row-check, meaning at least one of the hybridization or three median scale factors were outside the 0.4 to 2.5 range. Failing row-check indicates technical issues (e.g., clogs) with that particular sample that would not be fixed by running the sample again. Table 23 summarizes the data QC and samples removed from each ARIC dataset prior to analysis.

TABLE 23
Summary of Data QC for each timepoint in the dataset

| | Visit 2 | | Visit 3 | | Visit 5 | |
|---|---|---|---|---|---|---|
| | Samples | | Samples | | Samples | |
| Data | Removed | | Removed | | Removed | |
| Baseline | 11,779 | | 11,360 | | 5,281 | |
| (Clinical & | | | | | | |
| RFU Data) | | | | | | |
| Out-of-range | 17 | 44 | 36 | 72 | 0 | 27 |
| Outliers | 28 | | 41 | | 27 | |

| | | | |
|---|---|---|---|
| Analysis N | 11,735 | 11,288 | 5,254 |

For Aim 1 and Aim 2, samples from visit 2 and visit 3 were used. There were 10,048 visit 2 and visit 3 samples available for these aims. There were 19 prevalent lung cancer cases across both timepoints that were excluded from analysis, leaving 10,029 (99.811%) samples for analysis. Smoking behavior variables were created based on self-reported smoking exposure. Table 24 summarizes the smoking behavior variable.

The 274 samples missing or misclassified (10 individuals were missing visit 2 smoking status, 23 individuals missing visit 3 smoking status, and 241 individuals were misclassified reporting being a type of smoker at visit 2 and a never smoker at visit 3) samples were excluded from analysis, which left 9,755 (97.084%) for Aim 1 analysis. For the analysis of the smoking exposure variables, the “New Current Smoker” and “New Former Smokers” were combined into a “New Smoker” variable due to the small sample size in the “New Current Smoker” exposure group.

Of the 10,029 samples available for analysis, there were 624 individuals that were missing lung cancer diagnosis information leaving 9,405 (93.8%) samples for analysis for Aim 2.

TABLE 24
Summary of smoking behavior between visit 2 and visit 3.

| Visit 2 | Visit 3 | | | | |
|---|---|---|---|---|---|
| Smoking | Smoking | | | | |
| Status | Status | Smoking Behavior | | N | |
| Former | Former | Continued Quitter | | 3,536 | |
| Current | Current | Continued Smoker | | 1,644 | |
| Never | Never | Never Smoker | | 3,882 | |
| Never | Current | New Current | New | 5 | 199 |
| | | Smoker | Smoker | | |
| Never | Former | New Former | | 194 | |
| | | Smoker | | | |

| Current | Former | Quitter | 403 |
|---|---|---|---|
| Former | Current | Relapsed Smoker | 110 |
| Current/Former | Never | Misclassified or NA* | 274 |

*10 individuals were missing visit 2 smoking status, 23 individuals missing visit 3 smoking status, and 241 individuals were misclassified reporting being a type of smoker at visit 2 and a never smoker at visit 3.

### Aim 1 Results

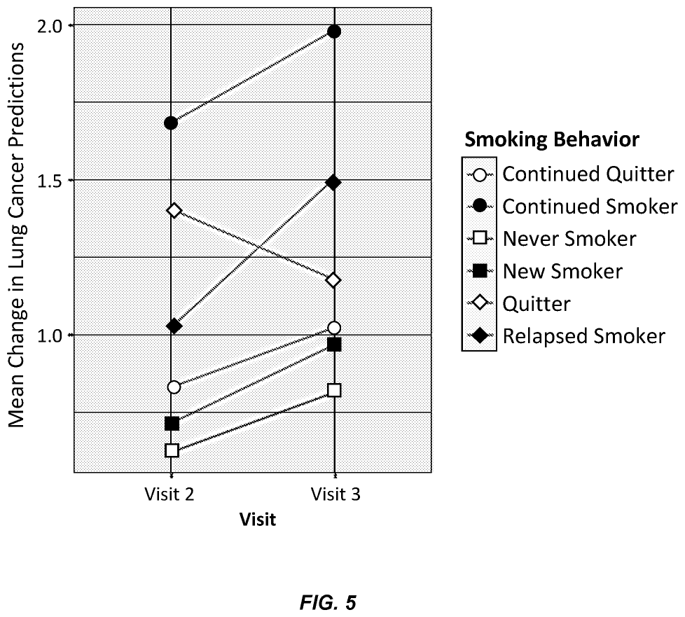

Aim 1 looked at changes in lung cancer susceptibility with changes or consistencies in smoking status between visit 2 and 3. All individuals with visit 2 and visit 3 samples without prevalent lung cancer (N =9,755) were used for this analysis. The mean difference in lung cancer predictions (FIG. 5), calculated as the difference in the mean lung cancer predictions at visit 3 (0.0111) and the mean lung cancer predictions at visit 2 (0.0092), were calculated (mean difference=0.0020; p<0.001). An ANOVA test was conducted to determine if there was a difference in lung cancer predictions between smoking status groups (p<0.001). To determine which smoking groups were statistically different from each other, post-hoc t-tests were conducted and summarized in Table 25.

TABLE 25
Summary of lung cancer predictions at visit 2 and visit 3 and change
in predictions between timepoints by smoking behavior group.

| | Mean Percent | Mean Percent | Absolute | Relative | |
|---|---|---|---|---|---|
| | LC | LC | Percent | Percent | |
| Smoking | Predictions | Predictions | Change in LC | Change in LC | p |
| Behavior | Visit 2 | Visit 3 | Predictions | Predictions | value |
| Quitter | 1.4055 | 1.1771 | −0.2285 | −16.3% | <0.001 |
| Continued | 0.8319 | 1.0251 | 0.1932 | 23.2% | <0.001 |
| Quitter | | | | | |
| Never | 0.6222 | 0.8161 | 0.1939 | 31.2% | <0.001 |
| Smoker | | | | | |
| New | 0.7162 | 0.9704 | 0.2542 | 35.5% | 0.002 |
| Smoker | | | | | |
| Continued | 1.6860 | 1.9863 | 0.3003 | 17.8% | <0.001 |
| Smoker | | | | | |
| Relapsed | 1.0255 | 1.5077 | 0.4822 | 47% | <0.001 |
| Smoker | | | | | |

TABLE 26
A summary of p-values from post-hoc t-tests (in the box) with
mean predictions for each smoking behavior group. The overall
ANOVA test is summarized in the last row of the table.

| | (p-value) | | | | |
|---|---|---|---|---|---|
| Continued | <0.001 | — | — | — | — |
| Smoker | | | | | |
| (N = 1,637) | | | | | |
| Never | 1.000 | <0.001 | — | — | — |
| Smoker | | | | | |
| (N = 3,880) | | | | | |
| New | 1.000 | 1.000 | 1.000 | — | — |
| Smoker | | | | | |
| (N = 199) | | | | | |
| Quitter | <0.001 | <0.001 | <0.001 | <0.001 | — |
| (N = 397) | | | | | |
| Relapsed | 0.0054 | 0.1764 | 0.0054 | 0.1725 | <0.001 |
| Smoker | | | | | |
| (N = 110) | | | | | |

| | |
|---|---|
| Overall | <0.001 |

The mean change in predictions between visit 2 and visit 3 for never smokers was 0.0019, continued smokers was 0.003, new smokers was 0.0025, quitters was-0.0023, relapsed smokers was 0.0048, and continued quitters was 0.0019 (summarized in Table 25).

Post-hoc t-tests showed a significant difference between continued quitters and continued smokers (difference=−0.0011; p<0.001), between continued smokers and never smokers (difference=0.0011; p<0.001), between continued quitters and quitters (difference=0.0042; p<0.001), between continued smokers and quitters (difference=0.0053; p<0.001), between never smokers and quitters (difference=0.0042; p<0.001), between new smokers and quitters (difference=0.0048; p<0.001), between continued quitters and relapsed smokers (difference=−0.0029; p=0.0054), between never smokers and relapsed smokers (difference=−0.0029; p=0.0054), and between quitters and relapsed smokers (difference=0.0071; p<0.001). There were no significant differences between continued quitters and never smokers, between continued quitters and new smokers, between continued smokers and new smokers, and between never smokers and new smokers (summarized in Table 26).

### Aim 2 Results

From the individuals with visit 2 and visit 3 individuals (N=9,755) that were used for Aim 1 analysis, there were 624 (6.6%) individuals that were missing lung cancer diagnosis information leaving 9,405 (93.4%) individuals with visit 2 and visit 3 samples for analysis. There were 313 individuals with an incident lung cancer diagnosis after visit 3 (until the end of follow up) and 63 individuals with an incident lung cancer diagnosis within 5 years of visit 3.

The change in lung cancer risk score between visit 2 and visit 3 was calculated. A t-test was used to determine if there was a significant difference between change in lung cancer risk score between visits 2 and 3 for those who were diagnosed with cancer compared who were never diagnosed with lung cancer (summarized Table 27).

TABLE 27
Summary of lung cancer predictions by timepoint and mean change between timepoints
with percent change in predictions between timepoints for all lung cancer individuals and
individuals who were diagnosed with lung cancer within 5 years of visit 3. P-value indicates
the results of the t-test of the change in lung cancer predictions between individuals
who were diagnosed with lung cancer after visit 3 and individuals who were not.

| | | | Mean | | | Mean | |
|---|---|---|---|---|---|---|---|
| | | | Change | | | Change | |
| | Mean at | Mean at | in LC | Mean at | Mean at | in LC | |
| | Visit 2 | Visit 3 | Predictions | Visit 2 | Visit 3 | Predictions | |
| Cohort | (%) | (%) | (% Change) | (%) | (%) | (% Change) | p- |
| (N_{lung cancer cases}) | Lung Cancer Dx = Yes | | | Lung Cancer Dx = No | | | value |
| Lung Cancer | 1.726 | 1.995 | 0.269 | 0.87 | 1.066 | 0.196 | 0.311 |
| (until end of | | | (13.5%) | | | (18.4%) | |
| follow up) | | | | | | | |
| (N = 313) | | | | | | | |
| Lung Cancer | 2.244 | 2.560 | 0.316 | 0.89 | 1.087 | 0.197 | 0.615 |
| (within 5 years) | | | (12.3%) | | | (18.1%) | |
| (N = 63) | | | | | | | |

Lung cancer visit 3 predictions were looked at in more detail in individuals that developed lung cancer in ≤ 5 years compared to individuals who developed lung cancer in >5 years (these two groups are mutually exclusive). Of the 313 individuals who developed lung cancer at visit 3, 63 individuals developed lung cancer within 5 years and 250 individuals developed lung cancer after 5 years. A t-test was used to compare these lung cancer predictions and are summarized in Table 28.

TABLE 28
Summary of lung cancer predictions in individuals who developed lung
cancer ≤5 years and >5 years (mutually exclusive groups) and
results of t-test (p-value) comparing these two groups at visit 3.

| Cohort | N | Mean Predictions | p value |
|---|---|---|---|
| Lung Cancer | 63 | 0.026 | 0.016 |
| Diagnosis ≤ 5 years | | | |
| Lung Cancer | 250 | 0.019 | |
| Diagnosis > 5 years | | | |

To determine if the changes in lung cancer risk predictions were predictive of time-to-cancer-diagnosis, concordance was measured. For this analysis, there were two (0.02%) individuals (lung cancer diagnosis=“N”) who had 0 days of follow-up time and were excluded from this analysis (analysis N=9,403). Table 29 summarizes the concordance analysis and shows that the changes in lung cancer predictions are not predictive (lung cancer all concordance=0.550; lung cancer 5-year concordance=0.556) of time-to-cancer-diagnosis.

TABLE 29
Summary of lung cancer concordance.

| Cohort | Concordance |
|---|---|
| Lung Cancer | 0.550 |
| (until end of follow up) | |
| Lung Cancer | 0.566 |
| (within 5 years) | |

### Aim 3 Results

From the individuals who had visit 5 samples (5,254), there were 300 (5.7%) individuals who were missing lung cancer diagnosis information and were excluded from this analysis. There were 9,454 (94.3%) samples for analysis. Samples from Aim 2 were used for the Aim 3 analysis.

A t-test was conducted to determine if there is a significant difference in lung cancer risk predictions between individuals who had prevalent lung cancer and individuals who did not. Table 30 and Table 31 summarize this analysis at visit 3 and visit 5, respectively, and show that there is a significant difference (p=0.003 and p=0.005 respectively) between individuals who have prevalent lung cancer (mean predictions visit 3=0.24; mean predictions visit 5=0.33) and individuals do not have prevalent lung cancer (mean predictions visit 3=0.011; mean predictions visit 5=0.021). There was one individual at visit 3 that had follow-up data at visit 5.

TABLE 30
Summary of t-test comparisons of lung cancer risk scores in ARIC
visit 3 participants with and without prevalent lung cancer.

| Prevalent Lung | | Mean Lung Cancer Prediction | p |
|---|---|---|---|
| Cancer | N | (Absolute Probability) | value |
| Yes | 20 | 0.24 | 0.003 |
| No | 11,268 | 0.011 | |

TABLE 31
Summary of t-test comparisons of lung cancer risk scores in ARIC
visit 5 participants with and without prevalent lung cancer.

| Prevalent Lung | | Mean Lung Cancer Prediction | p |
|---|---|---|---|
| Cancer | N | (Absolute Probability) | value |
| Y | 44 | 0.033 | 0.001 |
| N | 5,210 | 0.021 | |

### REFERENCES

All references listed below, or anywhere else throughout this description, are hereby incorporated by reference herein in their entireties:

1. Joshu et al. “Enhancing the Infrastructure of the Atherosclerosis Risk in Communities (ARIC) Study for Cancer Epidemiology Research: ARIC Cancer.” Cancer Epidemiol Biomarkers Prev 2018;27:295-305.

2. Tammemagi, et al. “Selection criteria for lung-cancer screening.” N Engl J Med 2013;368:728-36.

3. Siegel et al. “Cancer Statistics, 2021.” CA Cancer J Clin 2021;71:7-33.

4. “About Lung Cancer”, American Cancer Society. Available online at https://www.cancer.org/cancer/lung-cancer/about/.

5. “Cancer Stat Facts: Lung and Bronchus Cancer” National Cancer Institute. Surveillance, Epidemiology, and End Results Program. Available online at https://seer.cancer.gov/statfacts/html/lungb.html.

6. “Non-Small Cell Lung Cancer Treatment (PDQ®)-Patient Version” (August 2021) National Cancer Institute. Available online at https://www.cancer.gov/types/lung/patient/non-small-cell-lung-treatment-pdq#_118

7. “Lung Cancer Among People Who Never Smoked” (November 2020) Center for Disease Control and Prevention. Available online at https://www.cdc.gov/cancer/lung/nonsmokers/index.htm

8. Bruder et al. “Estimating lifetime and 10-year risk of lung cancer.” Prev Med Rep 2018; 11:125-30.

9. Samet J. M. “Health benefits of smoking cessation.” Clin Chest Med 1991; 12:669-79.

10. Force USPST, et al. “Screening for Lung Cancer: US Preventive Services Task Force Recommendation Statement.” JAMA 2021;325:962-70.

11. National Lung Screening Trial Research Team. “Reduced lung-cancer mortality with low-dose computed tomographic screening.” N Engl J Med 2011;365:395-409.

12. “Lung CT Screening Reporting & Data System (Lung-RADS)” American College of Radiology, Available online at https://www.acr.org/Clinical-Resources/Reporting-and-Data-Systems/Lung-Rads.

13. “Lung Cancer Screening” (March 2021) Mayo Clinic. Available online at https://www.mayoclinic.org/tests-procedures/lung-cancer-screening/about/pac-20385024.

14. Zahnd et al. “Lung Cancer Screening Utilization: A Behavioral Risk Factor Surveillance System Analysis.” Am J Prev Med 2019;57:250-5.

15. Tammemagi M. C. “Application of risk prediction models to lung cancer screening: a review.” J Thorac Imaging 2015;30:88-100.

16. “SEER *Explorer” National Cancer Institute. Surveillance, Epidemiology, and End Results Program. Available online at https://seer.cancer.gov/explorer.

17. Fuentes et al. “Comprehension of Top 200 Prescribed Drugs in the US as a Resource for Pharmacy Teaching, Training and Practice.” Pharmacy (Basel) 2018;6.

18. Roman J. “On the ‘TRAIL’ of a Killer: MMP12 in Lung Cancer.” Am J Respir Crit Care Med. 2017 Aug. 1;196 (3): 262-264.

19. Mehan et al. “Validation of a blood protein signature for non-small cell lung cancer.” Clin Proteomics. 2014 Aug. 1;11 (1): 32. doi: 10.1186/1559-0275-11-32.

20. Umeda et al. “Surfactant protein D inhibits activation of non-small cell lung cancer-associated mutant EGFR and affects clinical outcomes of patients.” Oncogene. 2017 Nov. 16;36 (46): 6432-6445.

21. Fu et al. “A meta-analysis of influence of MSMB promoter rs10993994 polymorphisms on prostate cancer risk.” Eur Rev Med Pharmacol Sci. 2019 November;23 (21): 9295-9303.

22. Lv et al. “Diagnostic value of human epididymis protein 4 in malignant pleural effusion in lung cancer.” Cancer Biomark. 2019;26 (4): 523-528

23. Moore, et al. “HE4 (WFDC2) gene overexpression promotes ovarian tumor growth.” Sci Rep 4, 3574 (2014)

What is claimed is:

1. A method comprising:
a) measuring the level of PSP-94 protein and the level of at least one, two, three, four, five, or six proteins selected from the group consisting of MMP-12, SP-D, HE4, PH, FUT5 and CRLF1 in a sample from a human subject; and
b) identifying the human subject as being at risk for developing lung cancer based on the level of PSP-94 and the level of the at least one, two, three, four, five, or six proteins.

2. A method comprising:
a) measuring the level of PH protein and the level of at least one, two, three, four, five, or six proteins selected from the group consisting of MMP-12, SP-D, HE4, PSP-94, FUT5 and CRLF1 in a sample from a human subject; and
b) identifying the human subject as being at risk for developing lung cancer based on the level of PH and the level of the at least one, two, three, four, five, or six proteins.

3. A method comprising:
a) measuring the level of FUT5 protein and the level of at least one, two, three, four, five, or six proteins selected from the group consisting of MMP-12, SP-D, HE4, PSP-94, PH and CRLF1 in a sample from a human subject; and
b) identifying the human subject as being at risk for developing lung cancer based on the level of FUT5 and the level of the at least one, two, three, four, five, or six proteins.

4. A method comprising:
a) measuring the level of CRLF1 protein and the level of at least one, two, three, four, five, or six proteins selected from the group consisting of MMP-12, SP-D, HE4, PSP-94, PH and FUT5 in a sample from a human subject; and
b) identifying the human subject as being at risk for developing lung cancer based on the level of CRLF1 and the level of the at least one, two, three, four, five, or six proteins.

5. A method comprising:
a) contacting a sample from a human subject with a set of capture reagents, wherein each capture reagent has affinity for a different protein of the set of proteins comprising PSP-94 protein, and at least one, two, three, four, five, or six proteins selected from the group consisting of MMP-12, SP-D, HE4, PH, FUT5 and CRLF1; and
b) measuring the level of each protein of the set of proteins with the set of capture reagents.

6. A method comprising:
a) contacting a sample from a human subject with a set of capture reagents, wherein each capture reagent has affinity for a different protein of the set of proteins comprising PH protein, and at least one, two, three, four, five, or six proteins selected from the group consisting of MMP-12, SP-D, HE4, PSP-94, FUT5 and CRLF1; and
b) measuring the level of each protein of the set of proteins with the set of capture reagents.

7. A method comprising:
a) contacting a sample from a human subject with a set of capture reagents, wherein each capture reagent has affinity for a different protein of the set of proteins comprising FUT5 protein, and at least one, two, three, four, five, or six proteins selected from the group consisting of MMP-12, SP-D, HE4, PSP-94, PH and CRLF1; and
b) measuring the level of each protein of the set of proteins with the set of capture reagents.

8. A method comprising:
a) contacting a sample from a human subject with a set of capture reagents, wherein each capture reagent has affinity for a different protein of the set of proteins comprising CRLF1 protein, and at least one, two, three, four, five, or six proteins selected from the group consisting of MMP-12, SP-D, HE4, PSP-94, PH and FUT5; and
b) measuring the level of each protein of the set of proteins with the set of capture reagents.

9. The method of claim 1 or claim 5, wherein the method comprises measuring PSP-94 and MMP-12; PSP-94 and SP-D; PSP-94 and HE4; PSP-94 and PH; PSP-94 and FUT5; or PSP-94 and CRLF1.

10. The method of claim 1 or claim 5, wherein the method comprises measuring PSP-94, MMP-12 and SP-D; PSP-94, MMP-12 and HE4; PSP-94, MMP-12 and PH; PSP-94, MMP-12 and FUT5; PSP-94, MMP-12 and CRLF1; PSP-94, SP-D and HE4; PSP-94, SP-D and PH; PSP-94, SP-D and FUT5; PSP-94, SP-D and CRLF1; PSP-94, HE4 and PH; PSP-94, HE4 and FUT5; PSP-94, HE4 and CRLF1; PSP-94, PH and FUT5; PSP-94, PH and CRLF1; or PSP-94, FUT5 and CRLF1.

11. The method of claim 2 or claim 6, wherein the method comprises measuring PH and MMP-12; PH and SP-D; PH and HE4; PH and PSP-94; PH and FUT5; or PH and CRLF1.

12. The method of claim 2 or claim 6, wherein the method comprises measuring PH, MMP-12 and SP-D; PH, MMP-12 and HE4; PH, MMP-12 and PSP-94; PH, MMP-12 and FUT5; PH, MMP-12 and CRLF1; PH, SP-D and HE4; PH, SP-D and PSP-94; PH, SP-D and FUT5; PH, SP-D and CRLF1; PH, HE4 and PSP-94; PH, HE4 and FUT5; PH, HE4 and CRLF1; PH, PSP-94 and FUT5; PH, PSP-94 and CRLF1; or PH, FUT5 and CRLF1.

13. The method of claim 3 or claim 7, wherein the method comprises measuring FUT5 and MMP-12; FUT5 and SP-D; FUT5 and HE4; FUT5 and PSP-94; FUT5 and PH; or FUT5 and CRLF1.

14. The method of claim 3 or claim 7, wherein the method comprises measuring FUT5, MMP-12 and SP-D; FUT5, MMP-12 and HE4; FUT5, MMP-12 and PSP-94; FUT5, MMP-12 and PH; FUT5, MMP-12 and CRLF1; FUT5, SP-D and HE4; FUT5, SP-D and PSP-94; FUT5, SP-D and PH; FUT5, SP-D and CRLF1; FUT5, HE4 and PSP-94; FUT5, HE4 and PH; FUT5, HE4 and CRLF1; FUT5, PSP-94 and PH; FUT5, PSP-94 and CRLF1; or FUT5, PH and CRLF1.

15. The method of claim 4 or claim 8, wherein the method comprises measuring CRLF1 and MMP-12; CRLF1 and SP-D; CRLF1 and HE4; CRLF1 and PSP-94; CRLF1 and PH; or CRLF1 and FUT5.

16. The method of claim 4 or claim 8, wherein the method comprises measuring CRLF1, MMP-12 and SP-D; CRLF1, MMP-12 and HE4; CRLF1, MMP-12 and PSP-94;CRLF1, MMP-12 and PH; CRLF1, MMP-12 and FUT5; CRLF1, SP-D and HE4; CRLF1, SP-D and PSP-94; CRLF1, SP-D and PH; CRLF1, SP-D and FUT5; CRLF1, HE4 and PSP-94; CRLF1, HE4 and PH; CRLF1, HE4 and FUT5; CRLF1, PSP-94 and PH; CRLF1, PSP-94 and FUT5; or CRLF1, PH and FUT5.

17. The method of claim 1 or claim 5, wherein the method comprises measuring PSP-94 and PH and at least one of the following proteins selected from MMP-12, SP-D, HE4, FUT5 and CRLF1.

18. The method of claim 1 or claim 5, wherein the method comprises measuring PSP-94 and FUT5 and at least one of the following proteins selected from MMP-12, SP-D, HE4, PH and CRLF1.

19. The method of claim 1 or claim 5, wherein the method comprises measuring PSP-94 and CRLF1 and at least one of the following proteins selected from MMP-12, SP-D, HE4, PH and FUT5.

20. The method of claim 2 or claim 6, wherein the method comprises measuring PH and FUT5 and at least one of the following proteins selected from MMP-12, SP-D, HE4, PSP-94 and CRLF1.

21. The method of claim 2 or claim 6, wherein the method comprises measuring PH and CRLF1 and at least one of the following proteins selected from MMP-12, SP-D, HE4, PSP-94 and FUT5.

22. The method of claim 3 or claim 7, wherein the method comprises measuring FUT5 and CRLF1 and at least one of the following proteins selected from MMP-12, SP-D, HE4, PSP-94 and PH.

23. A method comprising:
a) contacting a sample from a human subject with two capture reagents, wherein one capture reagent has affinity for a PSP-94 protein and the second capture reagent has affinity for a PH protein; and
b) measuring the level of each protein with the two capture reagents.

24. A method comprising:
a) contacting a sample from a human subject with two capture reagents, wherein one capture reagent has affinity for a PSP-94 protein and the second capture reagent has affinity for a FUT5 protein; and
b) measuring the level of each protein with the two capture reagents.

25. A method comprising:
a) contacting a sample from a human subject with two capture reagents, wherein one capture reagent has affinity for a PSP-94 protein and the second capture reagent has affinity for a CRLF1 protein; and
b) measuring the level of each protein with the two capture reagents.

26. A method comprising:
a) contacting a sample from a human subject with two capture reagents, wherein one capture reagent has affinity for a PH protein and the second capture reagent has affinity for a FUT5 protein; and
b) measuring the level of each protein with the two capture reagents.

27. A method comprising:
a) contacting a sample from a human subject with two capture reagents, wherein one capture reagent has affinity for a PH protein and the second capture reagent has affinity for a CRLF1 protein; and
b) measuring the level of each protein with the two capture reagents.

28. A method comprising:
a) contacting a sample from a human subject with two capture reagents, wherein one capture reagent has affinity for a FUT5 protein and the second capture reagent has affinity for a CRLF1 protein; and
b) measuring the level of each protein with the two capture reagents.

29. A method comprising:
a) measuring the level of PSP-94 and PH in a sample from a human subject; and
b) identifying the human subject as being at risk for developing lung cancer based on the level of PSP-94 and PH.

30. A method comprising:
a) measuring the level of PSP-94 and FUT5 in a sample from a human subject; and
b) identifying the human subject as being at risk for developing lung cancer based on the level of PSP-94 and FUT5.

31. A method comprising:
a) measuring the level of PSP-94 and CRLF1 in a sample from a human subject; and
b) identifying the human subject as being at risk for developing lung cancer based on the level of PSP-94 and CRLF1.

32. A method comprising:
a) measuring the level of PH and FUT5 in a sample from a human subject; and
b) identifying the human subject as being at risk for developing lung cancer based on the level of PH and FUT5.

33. A method comprising:
a) measuring the level of PH and CRLF1 in a sample from a human subject; and
b) identifying the human subject as being at risk for developing lung cancer based on the level of PH and CRLF1.

34. A method comprising:
a) measuring the level of FUT5 and CRLF1 in a sample from a human subject; and
b) identifying the human subject as being at risk for developing lung cancer based on the level of FUT5 and CRLF1.

35. A method comprising:
a) contacting a sample from a human subject with three capture reagents, wherein each of the three capture reagents has affinity for a protein selected from PSP-94, PH, and FUT5; and
b) measuring the level of each protein with the three capture reagents.

36. A method comprising:
a) contacting a sample from a human subject with three capture reagents, wherein each of the three capture reagents has affinity for a protein selected from PSP-94, PH, and CRLF1; and
b) measuring the level of each protein with the three capture reagents.

37. A method comprising:
a) contacting a sample from a human subject with three capture reagents, wherein each of the three capture reagents has affinity for a protein selected from PH, FUT5, and CRLF1; and
b) measuring the level of each protein with the three capture reagents.

38. A method comprising:
a) contacting a sample from a human subject with three capture reagents, wherein each of the three capture reagents has affinity for a protein selected from FUT5, CRLF1, and PSP-94; and
b) measuring the level of each protein with the three capture reagents.

39. A method comprising:
a) measuring the level of PSP-94, PH, and FUT5 in a sample from a human subject; and
b) identifying the human subject as being at risk for developing lung cancer based on the level of PSP-94, PH, and FUT5.

40. A method comprising:
a) measuring the level of PSP-94, PH, and CRLF1 in a sample from a human subject; and
b) identifying the human subject as being at risk for developing lung cancer based on the level of PSP-94, PH, and CRLF1.

41. A method comprising:
a) measuring the level of PH, FUT5, and CRLF1 in a sample from a human subject; and
b) identifying the human subject as being at risk for developing lung cancer based on the level of PH, FUT5, and CRLF1.

42. A method comprising:
a) measuring the level of FUT5; CRLF1, and PSP-94 in a sample from a human subject; and
b) identifying the human subject as being at risk for developing lung cancer based on the level of FUT5; CRLF1, and PSP-94.

43. The method of any one of claims 23-42, further comprising measuring the level of MMP-12 protein.

44. The method of any one of claims 23-43, further comprising measuring the level of SP-D protein.

45. The method of any one of claims 23-44, further comprising measuring the level of HE4 protein.

46. A method comprising:
a) measuring the level of at least three, four, five, six, or seven proteins selected from the group consisting of MMP-12, SP-D, HE4, PSP-94, PH, FUT5 and CRLF1 in a sample from a human subject; and
b) identifying the human subject as being at risk for developing lung cancer based on the level of the at least three, four, five, six, or seven proteins.

47. The method of claim 46, wherein the method comprises measuring MMP-12, SP-D and HE4; MMP-12, SP-D and PSP-94; MMP-12, SP-D and PH; MMP-12, SP-D and FUT5; MMP-12, SP-D and CRLF1; MMP-12, HE4 and PSP-94; MMP-12, HE4 and PH; MMP-12, HE4 and FUT5; MP-12, HE4 and CRLF1; MMP-12, PSP-94 and PH; MMP-12, PSP-94 and FUT5; MMP-12, PSP-94 and CRLF1; MMP-12, PH and FUT5; MMP-12, PH and CRLF1; MMP-12, FUT5 and CRLF1; SP-D, HE4 and PSP-94; SP-D, HE4 and PH; SP-D, HE4 and FUT5; SP-D, HE4 and CRLF1; SP-D, PSP-94 and PH; SP-D, PSP-94 and FUT5; SP-D, PSP-94 and CRLF1; SP-D, PH and FUT5; SP-D, PH and CRLF1; SP-D, FUT5 and CRLF1; HE4, PSP-94 and PH; HE4, PSP-94 and FUT5; HE4, PSP-94 and CRLF1; HE4, PH and FUT5; HE4, PH and CRLF1; HE4, FUT5 and CRLF1; PSP-94, PH and FUT5; PSP-94, PH and CRLF1; PSP-94, FUT5 and CRLF1; or PH, FUT5, and CRLF1.

48. The method of claim 46 or claim 47, further comprising measuring one or more of PSP-94, PH, FUT5, and CRLF1.

49. A method comprising:
a) contacting a sample from a human subject with a set of capture reagents, wherein each capture reagent has affinity for a different protein of the set of proteins comprising at least three, four, five, six, or seven proteins selected from the group consisting of MMP-12, SP-D, HE4, PSP-94, PH, FUT5 and CRLF1 in a sample from a human subject; and
b) measuring the level of each protein of the set of proteins with the set of capture reagents.

50. The method of claim 49, wherein the method comprises measuring MMP-12, SP-D and HE4; MMP-12, SP-D and PSP-94; MMP-12, SP-D and PH; MMP-12, SP-D and FUT5;
MMP-12, SP-D and CRLF1; MMP-12, HE4 and PSP-94; MMP-12, HE4 and PH; MMP-12, HE4 and FUT5; MP-12, HE4 and CRLF1; MMP-12, PSP-94 and PH; MMP-12, PSP-94 and FUT5; MMP-12, PSP-94 and CRLF1; MMP-12, PH and FUT5; MMP-12, PH and CRLF1;
MMP-12, FUT5 and CRLF1; SP-D, HE4 and PSP-94; SP-D, HE4 and PH; SP-D, HE4 and FUT5; SP-D, HE4 and CRLF1; SP-D, PSP-94 and PH; SP-D, PSP-94 and FUT5; SP-D, PSP-94 and CRLF1; SP-D, PH and FUT5; SP-D, PH and CRLF1; SP-D, FUT5 and CRLF1; HE4, PSP-94 and PH; HE4, PSP-94 and FUT5; HE4, PSP-94 and CRLF1; HE4, PH and FUT5; HE4, PH and CRLF1; HE4, FUT5 and CRLF1; PSP-94, PH and FUT5; PSP-94, PH and CRLF1; PSP-94, FUT5 and CRLF1; or PH, FUT5, and CRLF1.

51. The method of claim 49 or claim 50, further comprising measuring one or more of PSP-94, PH, FUT5 and CRLF1.

52. A method comprising:
a) measuring the level of MMP-12 protein, and the level of at least one, two, three, four, five, or six proteins selected from the group consisting of SP-D, HE4, PSP-94, PH, FUT5 and CRLF1 in a sample from a human subject; and
b) identifying the human subject as being at risk for developing lung cancer based on the level of MMP-12 and the level of the at least one, two, three, four, five, or six, proteins.

53. A method comprising:
a) measuring the level of SP-D protein, and the level of at least one, two, three, four, five, or six proteins selected from the group consisting of MMP-12, HE4, PSP-94, PH, FUT5 and CRLF1 in a sample from a human subject; and
b) identifying the human subject as being at risk for developing lung cancer based on the level of SP-D and the level of the at least one, two, three, four, five, six, seven, eight, or nine proteins.

54. A method comprising:
a) measuring the level of HE4 protein, and the level of at least one, two, three, four, five, or six proteins selected from the group consisting of MMP-12, SP-D, PSP-94, PH, FUT5 and CRLF1 in a sample from a human subject; and
b) identifying the human subject as being at risk for developing lung cancer based on the level of HE4 and the level of the at least one, two, three, four, five, six, seven, eight, or nine proteins.

55. A method comprising:
a) contacting a sample from a human subject with a set of capture reagents, wherein each capture reagent has affinity for a different protein of the set of proteins comprising MMP-12 protein, and at least one, two, three, four, five, or six proteins selected from the group consisting of SP-D, HE4, PSP-94, PH, FUT5 and CRLF1; and
b) measuring the level of each protein of the set of proteins with the set of capture reagents.

56. A method comprising:
a) contacting a sample from a human subject with a set of capture reagents, wherein each capture reagent has affinity for a different protein of the set of proteins comprising SP-D protein, and at least one, two, three, four, five, or six proteins selected from the group consisting of MMP-12, HE4, PSP-94, PH, FUT5 and CRLF1; and
b) measuring the level of each protein of the set of proteins with the set of capture reagents.

57. A method comprising:
a) contacting a sample from a human subject with a set of capture reagents, wherein each capture reagent has affinity for a different protein of the set of proteins comprising HE4 protein, and at least one, two, three, four, five, or six proteins selected from the group consisting of MMP-12, SP-D, PSP-94, PH, FUT5 and CRLF1; and
b) measuring the level of each protein of the set of proteins with the set of capture reagents.

58. The method of any one of claims 5-28, 36-38, 49-51, and 55-57, wherein the set of capture reagents is selected from aptamers, antibodies and a combination of aptamers and antibodies.

59. The method of any one of the preceding claims, wherein the measuring is performed using mass spectrometry, an aptamer based assay and/or an antibody based assay.

60. The method of claim 58 or claim 59, wherein the level of each biomarker protein measured is determined from a relative florescence unit (RFU) or a protein concentration.

61. The method of any one of the preceding claims, wherein the sample is selected from blood, plasma, serum or urine.

62. The method of any one of the preceding claims, wherein the protein levels are used to identify a human subject as being at risk for developing lung cancer.

63. The method of claim 62, wherein the risk for developing lung cancer is within a 5 year period.

64. The method of claim 62 or claim 63, wherein the risk for developing lung cancer is within a period of 1 year, 2 years, 3 years, 4 years, 5 years, 6 years, 7 years, 8 years, 9 years, or 10 years.

65. The method of any one of the preceding claims, wherein the human subject is a current smoker or a former smoker.

66. The method of any one of the preceding claims, wherein the subject has lung cancer.

67. The method of any one of the preceding claims, wherein the method provides an area under the curve (AUC) of 0.62, 0.67, 0.68, 0.68, 0.70, 0.71, 0.72, 0.73, 0.74, 0.75, 0.76, or above.

68. The method of any one of the preceding claims, wherein the method provides an area under the curve (AUC) from about 0.6 to about 0.8, from about 0.61 to about 0.78, from about 0.62 to about 0.76, from about 0.62 to about 0.68, from about 0.67 to about 0.72, from about 0.69 to about 0.74, from about 0.71 to about 0.74, from about 0.73 to about 0.76, or from about 0.74 to about 0.76.

69. The method of any one of the preceding claims, wherein predicting the risk for developing lung cancer is based on input of the levels of the measured proteins in a statistical model.

70. The method of claim 69, wherein the predicting comprises analyzing the levels of the measured proteins using an Accelerated Failure Time (AFT) Weibull survival model.

71. The method of any one of the preceding claims, further comprising performing a diagnostic screening.

72. The method of claim 71, wherein the diagnostic screening is selected from low dose computed tomography (LDCT), chest radiography, and sputum cytology.

73. The method of any one of the preceding claims, wherein the method comprises predicting the risk for developing lung cancer for the purpose of determining a medical insurance premium or life insurance premium.

74. The method of claim 73, wherein the method further comprises determining coverage or premium for medical insurance or life insurance.

75. The method of any one of claims 1-72, wherein the method further comprises using information resulting from the method to predict and/or manage the utilization of medical resources.

76. The method of any one of claims 1-72, wherein the method further comprises using information resulting from the method to enable a decision to acquire or purchase a medical practice, hospital, or company.

77. A kit comprising N protein capture reagents, wherein N is at least 1, at least 2, at least 3, at least 4, at least 5, at least 6, or at least 7, and wherein at least one of the N protein capture reagents specifically binds to a protein selected from PSP-94, MMP-12, SP-D, HE4, PH, FUT5 and CRLF1.

78. The kit of claim 77, wherein Nis at least two and at least one to the two N protein capture reagents specifically binds to the protein selected from PSP-94, MMP-12, SP-D, HE4, PH, FUT5 and CRLF1.

79. The kit of claim 77 or 78, wherein N is 2 to 7, or N is 3 to 7, or N is 4 to 7, or N is 5 to 7, or N is 6 to 7.

80. The kit of any one of claims 77-79, wherein N is 2, N is 3, N is 4, N is 5, N is 6, or N is 7.

81. The kit of any one of claims 77-80, wherein each of the N protein capture reagents specifically binds to a different biomarker protein.

82. The kit of any one of claims 77-81, wherein each of the N protein capture reagents specifically binds to a protein selected from PSP-94, MMP-12, SP-D, HE4, PH, FUT5 and CRLF1.

83. The kit of any one of claims 77-81, wherein two of the N protein capture reagents specifically bind PSP-94 and MMP-12; or two of the N protein capture reagents specifically bind PSP-94 and SP-D; or two of the N protein capture reagents specifically bind PSP-94 and HE4; or two of the N protein capture reagents specifically bind PSP-94 and PH; or two of the N protein capture reagents specifically bind PSP-94 and FUT5; or two of the N protein capture reagents specifically bind PSP-94 and CRLF1.

84. The kit of any one of claims 77-81, wherein three of the N protein capture reagents specifically bind PSP-94, MMP-12 and SP-D; or three of the N protein capture reagents specifically bind PSP-94, MMP-12 and HE4; or three of the N protein capture reagents specifically bind PSP-94, MMP-12 and PH; or three of the N protein capture reagents specifically bind PSP-94, MMP-12 and FUT5; or three of the N protein capture reagents specifically bind PSP-94, MMP-12 and CRLF1; or three of the N protein capture reagents specifically bind PSP-94, SP-D and HE4; or three of the N protein capture reagents specifically bind PSP-94, SP-D and PH; or three of the N protein capture reagents specifically bind PSP-94, SP-D and FUT5; or three of the N protein capture reagents specifically bind PSP-94, SP-D and CRLF1; or three of the N protein capture reagents specifically bind PSP-94, HE4 and PH; PSP-94, HE4 and FUT5; or three of the N protein capture reagents specifically bind PSP-94, HE4 and CRLF1; or three of the N protein capture reagents specifically bind PSP-94, PH and FUT5; or three of the N protein capture reagents specifically bind PSP-94, PH and CRLF1; or three of the N protein capture reagents specifically bind PSP-94, FUT5 and CRLF1.

85. The kit of any one of claims 77-81, wherein two of the N protein capture reagents specifically bind PH and MMP-12; or two of the N protein capture reagents specifically bind PH and SP-D; or two of the N protein capture reagents specifically bind PH and HE4; or two of the N protein capture reagents specifically bind PH and PSP-94; PH and FUT5; or two of the N protein capture reagents specifically bind PH and CRLF1.

86. The kit of any one of claims 77-81, wherein three of the N protein capture reagents specifically bind PH, MMP-12 and SP-D; or three of the N protein capture reagents specifically bind PH, MMP-12 and HE4; or three of the N protein capture reagents specifically bind PH, MMP-12 and PSP-94; or three of the N protein capture reagents specifically bind PH, MMP-12 and FUT5; or three of the N protein capture reagents specifically bind PH, MMP-12 and CRLF1; or three of the N protein capture reagents specifically bind PH, SP-D and HE4; or three of the N protein capture reagents specifically bind PH, SP-D and PSP-94; or three of the N protein capture reagents specifically bind PH, SP-D and FUT5; or three of the N protein capture reagents specifically bind PH, SP-D and CRLF1; or three of the N protein capture reagents specifically bind PH, HE4 and PSP-94; or three of the N protein capture reagents specifically bind PH, HE4 and FUT5; or three of the N protein capture reagents specifically bind PH, HE4 and CRLF1; or three of the N protein capture reagents specifically bind PH, PSP-94 and FUT5; PH, PSP-94 and CRLF1; or three of the N protein capture reagents specifically bind PH, FUT5 and CRLF1.

87. The kit of any one of claims 77-81, wherein two of the N protein capture reagents specifically bind FUT5 and MMP-12; or two of the N protein capture reagents specifically bind FUT5 and SP-D; or two of the N protein capture reagents specifically bind FUT5 and HE4; or two of the N protein capture reagents specifically bind FUT5 and PSP-94; or two of the N protein capture reagents specifically bind FUT5 and PH; or two of the N protein capture reagents specifically bind FUT5 and CRLF1.

88. The kit of any one of claims 77-81, wherein three of the N protein capture reagents specifically bind FUT5, MMP-12 and SP-D; or three of the N protein capture reagents specifically bind FUT5, MMP-12 and HE4; or three of the N protein capture reagents specifically bind FUT5, MMP-12 and PSP-94; or three of the N protein capture reagents specifically bind FUT5, MMP-12 and PH; or three of the N protein capture reagents specifically bind FUT5, MMP-12 and CRLF1; or three of the N protein capture reagents specifically bind FUT5, SP-D and HE4; or three of the N protein capture reagents specifically bind FUT5, SP-D and PSP-94; or three of the N protein capture reagents specifically bind FUT5, SP-D and PH; or three of the N protein capture reagents specifically bind FUT5, SP-D and CRLF1; or three of the N protein capture reagents specifically bind FUT5, HE4 and PSP-94; or three of the N protein capture reagents specifically bind FUT5, HE4 and PH; or three of the N protein capture reagents specifically bind FUT5, HE4 and CRLF1; or three of the N protein capture reagents specifically bind FUT5, PSP-94 and PH; or three of the N protein capture reagents specifically bind FUT5, PSP-94 and CRLF1; or three of the N protein capture reagents specifically bind FUT5, PH and CRLF1.

89. The kit of any one of claims 77-81, wherein two of the N protein capture reagents specifically bind CRLF1 and MMP-12; or two of the N protein capture reagents specifically bind CRLF1 and SP-D; or two of the N protein capture reagents specifically bind CRLF1 and HE4; or CRLF1 and PSP-94; or two of the N protein capture reagents specifically bind CRLF1 and PH; or two of the N protein capture reagents specifically bind CRLF1 and FUT5.

90. The kit of any one of claims 77-81, wherein three of the N protein capture reagents specifically bind CRLF1, MMP-12 and SP-D; or three of the N protein capture reagents specifically bind CRLF1, MMP-12 and HE4; or three of the N protein capture reagents specifically bind CRLF1, MMP-12 and PSP-94; or three of the N protein capture reagents specifically bind CRLF1, MMP-12 and PH; or three of the N protein capture reagents specifically bind CRLF1, MMP-12 and FUT5; or three of the N protein capture reagents specifically bind CRLF1, SP-D and HE4; or three of the N protein capture reagents specifically bind CRLF1, SP-D and PSP-94; or three of the N protein capture reagents specifically bind CRLF1, SP-D and PH; or three of the N protein capture reagents specifically bind CRLF1, SP-D and FUT5; or three of the N protein capture reagents specifically bind CRLF1, HE4 and PSP-94; or three of the N protein capture reagents specifically bind CRLF1, HE4 and PH; or three of the N protein capture reagents specifically bind CRLF1, HE4 and FUT5; or three of the N protein capture reagents specifically bind CRLF1, PSP-94 and PH; or three of the N protein capture reagents specifically bind CRLF1, PSP-94 and FUT5; or three of the N protein capture reagents specifically bind CRLF1, PH and FUT5.

91. A kit comprising N protein capture reagents, wherein the kit comprises protein capture reagents for carrying out the methods any one of claims 1-76.

92. The kit of any one of claims 77-91, wherein each of the N biomarker protein capture reagents is an antibody or an aptamer.

93. The kit of claim 92, wherein each biomarker protein capture reagent is an aptamer.

94. The kit of any one of claims 77-93, for use in detecting the N biomarker proteins in a sample from a subject.

95. The kit of claim 94, for use in predicting risk of a subject for developing lung cancer.

96. The kit of claim 95, wherein the subject has lung cancer.
